(19) United States
(12) Patent Application Publication
(10) Pub. No.: US 2006/0030537 A1
(43) Pub. Date: Feb. 9, 2006

(54) Ceramide kinase and uses thereof

(76) Inventors: Charles E. Chalfant, Petersburg, VA (US); Yusuf A. Hannun, Sullivans Island, SC (US); Benjamin J. Pettus, N. Charleston, SC (US); Alicja Bielawska, Charleston, SC (US)

Correspondence Address: JONES DAY, 222 EAST 41ST ST, NEW YORK, NY, 10017, US

(21) Appl. No.: 11/179,958

(22) Filed: Jul. 11, 2005

Related U.S. Application Data
(60) Provisional application No. 60/586,909, filed on Jul. 9, 2004.

(51) Int. Cl. A61K 48/00; A61K 31/739; C07H 21/02

(52) U.S. Cl. 514/44; 514/54; 536/23.1; 536/53

(57) ABSTRACT

The invention relates to a method of inhibiting the production of ceramide-1-phosphate in a cell by delivering to the cell a ceramide kinase antagonist. The invention also relates to a method of treating a condition related to activation of phospholipase A2 in a subject by administering to the subject a pharmaceutical composition comprising a ceramide kinase antagonist. The invention further relates to the use of ceramide kinase in drug screening assays. The invention also encompasses compounds that inhibit the production of ceramide-1-phosphate by ceramide kinase.

This application claims priority to U.S. provisional application Ser. No. 60/586,909, filed Jul. 9, 2004, which is incorporated by reference herein in its entirety.

This invention was made with Government support under grant number CA87584 awarded by the National Institutes of Health. The United States Government has certain rights in the invention.

### 1. FIELD OF THE INVENTION

The invention relates to a method of inhibiting the production of ceramide-1-phosphate in a cell by delivering to the cell a ceramide kinase antagonist. The invention also relates to a method of treating a condition relating to the activation of phospholipase A2 in a subject by administering to the subject a pharmaceutical composition comprising a ceramide kinase antagonist. The invention further relates to the use of ceramide kinase in drug screening assays for anti-inflammatory compounds. The invention also encompasses compounds that inhibit the production of ceramide-1-phosphate by modulating ceramide kinase.

### 2. BACKGROUND OF THE INVENTION

Eicosanoids are synthesized de novo from arachidonic acid (AA) released from membranes in response to agonists such as cytokines and growth factors. The production of AA is the initial rate-limiting step in eicosanoid biosynthesis; thus, the regulation of phospholipases, specifically phospholipase A₂, (“PLA₂”) is of key importance in this pathway (Clark, J. D., Schievella, A. R., Nalefski, E. A., and Lin, L. L. (1995) J. Lipid. Media.t Cell Signal. 12, 83-117; Funk, C. D. (2001) Science 294,1871-1875). At the Golgi apparatus, endoplasmic reticulum, and nuclear membrane, PLA₂ hydrolyzes membrane phospholipids to produce AA, beginning eicosanoid biosynthesis (Clark, J. D., Schievella, A. R., Nalefski, E. A., and Lin, L. L. (1995) J. Lipid. Media.t Cell Signal. 12, 83-117; Funk, C. D. (2001) Science 294,1871-1875). The AA produced is utilized by either lipoxygenases to produce leukotrienes or by cyclooxygenases (COX) to produce prostanoids (Clark, J. D., Schievella, A. R., Nalefski, E. A., and Lin, L. L. (1995) J. Lipid. Media.t Cell Signal. 12, 83-117; Funk, C. D. (2001) Science 294,1871-1875).

The COX-2 pathway of prostanoid synthesis has been established as an important therapeutic target for the treatment of inflammatory disorders (Williams, C. S., Mann, M., and DuBois, R. N. (1999) Oncogene 18, 7908-7916). More recently, it has been found that inhibitors of COX-2 reduce the growth and metastasis capabilities of some cancers. Specifically, in mouse models of familial adenomatous polyposis, it has been shown that crosses with knockout mice of either cytosolic phospholipase A₂ or COX-2 reduced the size and metastatic capability of tumors (Oshima, M., Dinchuk, J. E., Kargman, S. L., Oshima, H., Hancock, B., Kwong, E., Trzaskos, J. M., Evans, J. F., and Taketo, M. M. (1996) Cell 87, 803-809; Takaku, K., Sonoshita, M., Sasaki, N., Uozumi, N., Doi, Y., Shimizu, T., and Taketo, M. M. (2000) J. Biol. Chem. 275, 34013-34016; Yamauchi, T., Watanabe, M., Hasegawa, H., Nishibori, H., Ishii, Y., Tatematsu, H., Yamamoto, K., Kubota, T., and Kitajima, M. (2003) Anticancer Res. 23, 245-9). However, despite the value of COX-2 inhibitors for the treatment of disease states, inhibition of COX-2 is unable to reduce AA release that may lead to excess production of leukotrienes and thromboxanes, and thus, side effects. In addition, AA production leads to glutathione depletion and reactive oxygen species generation. For these reasons, PLA₂ as an earlier therapeutic target in the pathway of eicosanoid production is of increased importance. Consequently, identification of components in the pathways proximal to PLA₂ activation is of key importance and high priority.

The main component of the venom from Loxosceles reclusa (brown recluse spider) is the enzyme sphingomyelinase D (SMase D) (Kurpiewski, G., Forrester, L. J., Barrett, J. T., and Campbell, B. J. (1981) Biochim. Biophys. Acta 678, 467-476) which hydrolyzes sphingomyelin to produce ceramide-l-phosphate (C-1-P). The pathology of a wound generated from the bite of this spider consists of an intense inflammatory response mediated by AA and prostaglandins (Stibich, A. S., and Schwartz, R. A. (2001) eMedicine Journal 2(7); Arnold, T. (2001) eMedicine Journal 2(9); Rees, R. S., Gates, C., Timmons, S., Des Prez, R. M., and King, L. E., Jr. (1988) Toxicol. 26, 1035-1045). The production of endogenous C-1-P by the action of SMase D raised the intriguing possibility that C-1-P may act as an endogenous and proximal activator of PLA₂ and the subsequent inflammatory response mediated by prostaglandins.

Despite the importance of prostaglandins, little is known about the regulation of prostanoid synthesis proximal to the activation of cytosolic phospholipase A₂ (cPLA₂), the initial rate-limiting step. Thus, there is a need for understanding the regulation of prostanoid synthesis and phospholipase A2 which provides an opportunity to identify a drug target.

### 3. SUMMARY

In this invention, the present inventors discovered that ceramide-1-phosphate (C-1-P) is a specific and potent inducer of arachidonic acid (AA) and prostanoid synthesis in cells. The inventors also demonstrate that two well-established activators of AA release and prostanoid synthesis, the cytokine, IL-1β, and the calcium ionophore, A23187, induce an increase in C-1-P levels within the relevant time-frame of AA release. Furthermore, the enzyme responsible for the production of C-1-P in mammalian cells, ceramide kinase, was activated in response to IL-1β and A23187. RNAi targeted to ceramide kinase specifically downregulated ceramide kinase mRNA and activity with concomitant decrease of AA release in response to IL-1β and A23187. Downregulation of ceramide kinase had no effect on AA release induced by exogenous C-1-P. Collectively, the inventors discovered that ceramide kinase, via the formation of C-1-P, is an upstream modulator of PLA₂ activation. The invention is directed to previously unknown roles for ceramide kinase and its product, C-1-P, in AA release and production of eicosanoids, and its exploitation as a potential new target to block inflammatory responses.

In one embodiment, the invention provides methods for screening compounds that modulate the activity of ceramide kinase.

The present invention is also directed to a method of treating a condition, disease or disorder associated with arachidonic acid release or phospholipase A2 activation in an animal. The method comprises administering to an animal a compound that inhibits ceramide kinase activity in an amount sufficient to inhibit arachidonic acid release or phospholipase A2 activation. In specific embodiments, the present invention is directed to treatment of inflammation, cardiovascular disorder, and rheumatoid arthritis.

In another embodiment, the present invention is directed to a method of inhibiting the formation of ceramide-1-phosphate in a cell. The method comprises administering an effective amount of a ceramide kinase antagonist, such as an antibody to ceramide kinase, antisense nucleic acid, or RNAi molecules.

The invention is also directed to a method for identifying compounds that modulate ceramide kinase gene expression. The method comprises contacting a test compound with a cell or cell lysate comprising a ceramide kinase expression construct; and detecting the transcription or translation of the nucleotide sequence of ceramidase.

The invention is also directed to a method for identifying compounds that modulate ceramide kinase gene expression. The method comprises contacting a test compound with a cell or cell lysate containing a reporter gene operatively associated with the regulatory element of a ceramide kinase gene; and detecting expression of the reporter gene product.

The invention further provides a method of identifying a compound that modulates the activity of ceramide kinase. The method comprising contacting ceramide kinase or a functional fragment thereof with a compound under conditions conducive to binding between the compound and the enzyme, and determining the activity of the ceramide kinase.

The invention is also directed to a method for identifying compounds that modulate the activity of ceramide kinase gene product or homolog of ceramide kinase gene product. The method comprises contacting a test compound with an organism or a cell containing ceramide kinase gene product or homolog of ceramide kinase; and comparing the phenotype of the organism or cell with the phenotype of organism or cell that did not contact the test compound, wherein a change in phenotype indicates that the test compound is capable of modulating the activity of ceramide kinase gene product or homolog of ceramide kinase gene product.

Also encompassed are ceramide kinase antagonists that inhibit the activity of ceramide kinase such as antibodies to ceramide kinase, RNAi molecules of ceramide kinase, antisense ceramide kinase nucleic acids, and other compounds that modulate ceramide kinase gene expression or ceramide kinase activity that can be used for drug screening, and/or treatment of diseases and disorders, including but not limited to inflammation, cardiovascular disorder, asthma, and rheumatoid arthritis. Also encompassed are transgenic animals that do not express the ceramide kinase in certain cells of the transgenic animals

### 4. BRIEF DESCRIPTION OF DRAWINGS

FIGS. 1A-1D. The effects of natural ceramide-1-phosphate on AA release (FIGS. 1A and 1B) and prostaglandin E₂ (“PGE₂”) synthesis (FIGS. 1C and 1D) are time-dependent. A549 cells (5×10⁴) in 24-well plates were labeled overnight with 5 μCi/mL [³H]-arachidonic acid (5 nM). Cells were washed and placed in DMEM supplemented with 2% fetal bovine serum for two hours. Cells were then treated with 2.5 μM D-e-C₁₆ ceramide-1-phosphate (solubilized in 98% ethanol/2% dodecane) for the indicated times. Results were controlled for equivalent number of cells quantified by MTT assay and by verification of total AA labeling by scintillation counting. The results are presented as DPM of ³H-arachidonic acid per mL of media for vehicle control (H) and C-1-P (2.5 μM) (J). In parallel experiments, the synthesis of PGE₂ was assayed, and the results are presented as pg of PGE₂ per mL of media controlled for equivalent number of cells by MTT assay for vehicle control (H) and C-1-P (2.5 μM) (J). Data presented in this figure are representative of six separate determinations on 3 separate occasions.

FIGS. 2A-2B. The effects of ceramide-1-phosphate on AA release (FIG. 2A) and PGE₂ synthesis (FIG. 2B) are dose-dependent. As described in FIG. 1, A549 cells (5×10⁴) were labeled overnight with 5 μCi/mL [³H]-arachidonic acid (5 nM). Cells were washed and placed in DMEM supplemented with 2% fetal bovine serum for two hours. Cells were treated with various concentrations of D-e-C₁₆ ceramide-1-phosphate for 8 hours. The results are presented as DPM of ³H-arachidonic acid per mL for equivalent number of cells by MTT assay. In parallel experiments, PGE₂ synthesis was assayed, and results are presented as pg of PGE₂ per mL of media controlled for equivalent number of cells by MTT assay. Data are representative of six separate determinations on 3 separate occasions.

FIGS. 3A-3B. FIG. 3A. The effects of ceramide-1-phosphate on TXB₂ synthesis. A549 cells (1×10⁵) were treated with D-e-C₁₆ ceramide-1-phosphate (1 μM) solubilized in 2% dodecane/98% ethanol (final concentration in treatments was 0.002% dodecane/0.098% ethanol (EtOH)) for the indicated times. The results are presented as % control of TXB₂ controlled for equivalent number of cells by MTT assay. Data are representative of 3 separate determinations on 2 separate occasions. FIG. 3B. The effects of ceramide-1-phosphate on AA release and PGE₂ synthesis in L929 fibroblasts. L929 cells (5×10⁴) were labeled overnight with 5 μCi/mL [³H]-arachidonic acid (5 nM). Cells were washed and placed in DMEM supplemented with 2% fetal bovine serum for two hours. Cells were then treated with 2.5 μM D-e-C₁₆ ceramide-1-phosphate (solubilized in 98% ethanol/2% dodecane) for the indicated times. The results are presented as % control of 3H-arachidonic acid release (B) and as % control of PGE₂ synthesis (J) controlled for equivalent number of cells by MTT assay. Data are representative of 3 separate determinations on 2 separate occasions.

FIGS. 4A-4C. The effects of ceramide-1-phosphate on AA release and PGE₂ synthesis are lipid specific. FIG. 4A. The effects of ceramide-1-phosphate on AA release are lipid specific. A549 cells (5×10⁴) were labeled overnight with 5 μCi/mL [³H]-arachidonic acid (5 nM), washed, and treated with 1 μM of the indicated lipid (di-oleoyl phosphatidic acid (PA), D-e-sphingosine-1-phosphate (S-1-P), D-e-C₁₆ ceramide (CER), D-e-C₁₆ ceramide-1-phosphate (C-1-P), and dioleoyl glycerol (DAG)) solubilized in 2% dodecane/98% EtOH (final concentration in treatments was 0.002% dodecane/0.098% EtOH) for 2 hours. The results are presented as DPM of ³H-arachidonic acid mL of media controlled for equivalent number of cells by MTT assay. Data are representative of six separate determinations on 3 separate occasions. FIG. 4B. The effects of long term ceramide treatment on AA release. A549 cells (5×10⁴) were labeled overnight with 5 μCi/mL [³H]-arachidonic acid (5 nM), washed, and treated with 1 PLM of D-e-C₁₆ ceramide (CER) or D-e-C₁₆ ceramide-1-phosphate (C-1-P) solubilized in 2% dodecane/98% EtOH for 8 hours. The results are presented as DPM of ³H-arachidonic acid mL of media controlled for equivalent number of cells by MTT assay. Data are representative of four separate determinations on 3 separate occasions. FIG. 4C. The effects of endogenously generated ceramide-1-phosphate on AA release. A549 cells (5×10⁴) were treated with either 0.5 U/mL of SMase D for 8 hours, 100 mU/mL of SMase C for 8.5 hours, or pre-treated for 30 minutes with 100 mU/mL of SMase C followed by the addition of 0.5 U/mL of SMase D for 8 hours. The results are presented as normalized DPM of ³H-arachidonic acid per mL of media controlled for equivalent number of cells by MTT assay. Data are representative of four separate determinations on two separate occasions.

FIGS. 5A-5C. IL-1β and calcium ionophore increase ceramide-1-phosphate production and ceramide kinase activity. FIGS. 5A and 5B. The effect of IL-1 and A23187 on C-1-P levels and AA liberation. A549 cells (2×10⁵) were labeled overnight with 10 μCi/mL [³H]-arachidonic acid (10 nM), washed, and then treated with either 2.5 ng/mL of IL-1β (FIG. 5A) or 1 μg/mL of A23187 (FIG. 5B) for the indicated times. Two hours prior to Bligh-Dyer extraction, cells were chased with 20 μM of D-e-C₆ NBD-ceramide (Matreya). At the appropriate times, media was taken for AA analysis, and lipids from the cells were extracted by the Bligh-Dyer method followed by analysis of NBD-C-1-P formation. Data are presented as % control of C-1-P levels (J) and AA release (B). Data are representative of three separate determinations on 2 separate occasions. FIG. 5C. IL-1β and calcium ionophore increase ceramide kinase activity. A549 cells (5×10⁵) were treated with either 2.5 ng/mL of IL-1β for 3.5 hours or 1 μg/mL of A23187 for 5 minutes. Cells were then washed, lysed in assay buffer, and assayed for ceramide kinase activity. Data is presented as % control of ceramide kinase activity (Arbitrary densitometry units of fluorescent NBD-C-1-P produced/min/mg of total cellular protein). Data are representative of three separate determinations on 2 separate occasions.

FIGS. 6A-6B. Ceramide kinase RNAi downregulates endogenous ceramide kinase activity and mRNA levels. FIG. 6A. The effect of ceramide kinase RNAi on endogenous ceramide kinase activity. A549 cells (5×10⁴) were transfected with the 21-nucleotide duplexes for ceramide kinase or scrambled control sequence. Following the 4 hour transfection, cells incubated in normal growth media for 26 hours. Cells were then washed, lysed in assay buffer, and assayed for ceramide kinase activity. Data are presented as % control ceramide kinase activity with control RNAi (Control) or ceramide kinase RNAi (CerK RNAi). Data are representative of three separate determinations on two separate occasions. FIG. 6B. The effect of ceramide kinase RNAi on the mRNA levels of ceramide kinase. Following the same procedure as outlined in FIG. 6A, total RNA was extracted and analyzed via logarithmic reverse-transcriptase polymerase chain reaction for human ceramide kinase and 18s ribosomal RNA. Data are presented as percent control after the densitometry of ceramide kinase mRNA was normalized to 18s ribosomal RNA levels with control RNAi (Control) or ceramide kinase RNAi (CerK RNAi). Data are representative of three separate determinations on two separate occasions.

FIGS. 7A-7D. Ceramide kinase regulates AA release in response to IL-1β and calcium ionophore. FIG. 7A. The effect of ceramide kinase RNAi on AA release in response to IL-1β. A549 cells (5×10⁴) were transfected with the 21-nucleotide duplexes for ceramide kinase or scrambled control sequence. Following the 4 hour transfection, cells were incubated overnight. Cells were washed and placed in DMEM supplemented with 2% fetal bovine serum for two hours. After 26 hours post-transfection, cells were treated with IL-1β (2.5 ng/mL). Data are presented as AA release (DPM) after 4 hours of treatment for control RNAi plus vehicle (B), ceramide kinase RNAi plus vehicle (J), control RNAi plus IL-1β (2.5 ng/mL) (H), and ceramide kinase RNAi plus IL-1β (2.5 ng/mL) (F). Data are representative of six separate determinations on three separate occasions. FIG. 7B. The effect of ceramide kinase RNAi on AA release in response to 5 minutes of treatment with A23187. A549 cells (5×10⁴) were transfected with the 21-nucleotide duplexes for ceramide kinase or scrambled control sequence. Following the 4 hour transfection, cells were labeled overnight with 5 μCi/mL [³H]-arachidonic acid (5 nM). Cells were washed and placed in DMEM supplemented with 2% fetal bovine serum for two hours. After 26 hours post-transfection, cells were treated with A23187 (1 μg/mL) for 5 minutes and assayed for AA release. Data are presented as AA release (DPM) for control RNAi plus vehicle (B), ceramide kinase RNAi plus vehicle (J), control RNAi plus A23187 (1 μg/mL) (H), and ceramide kinase RNAi plus A23187 (1 μg/mL) (F). Data are representative of six separate determinations on three separate occasions. FIG. 7C. The effect of ceramide kinase RNAi on AA release in response to 30 minutes of treatment with A23187. Following the same procedure as outlined in FIG. 7B for ceramide kinase RNAi, cells were treated with A23187 (1 μg/mL) for 30 minutes and assayed for AA release. Data are presented as AA release (DPM) for control RNAi plus vehicle (B), ceramide kinase RNAi plus vehicle (J), control RNAi plus A23187 (1 μg/mL) (H), and ceramide kinase RNAi plus A23187 (1 μg/mL) (F). Data are representative of six separate determinations on three separate occasions. FIG. 7D. C-1-P overcomes the inhibition of ceramide kinase RNAi on AA release. Following the same procedure as outlined in FIG. 7B, cells transfected with control RNAi (Control) and ceramide kinase RNAi (CerK RNAi) were treated with C-1-P (2.5 μM) for 5 hours and AA release (DPM) measured. Data are representative of three separate determinations on 2 separate occasions.

FIG. 8. Ceramide kinase inhibitors. (2R,3S,4E) L-erythro-C6-ceramide and (2R,3S,4E) L-erythro-urea-C6-ceramide

### 5. DETAILED DESCRIPTION OF THE INVENTION

The present invention is based on the discovery that ceramide kinase mediates cytokine- and calcium ionophore-induced arachidonic acid release. The role for ceramide kinase and its product ceramide-1-phosphate in arachidonic acid release and production of eicosanoids was previously unknown. Accordingly, the invention provides methods for the prevention and/or treatment of inflammation and diseases caused by acute or choronic inflammation, such as but not limited to rheumatoid arthritis, atherosclerosis, lung inflammation developed by smokers that is linked to hyperactive nerves. The invention contemplates using ceramide kinase antagonists, such as but not limited to, small organic molecules, ceramide analogs, antibodies, antisense nucleic acids, RNAi molecules, ribozymes, to modulate the expression of ceramide kinase gene and/or the activity of ceramide kinase gene product.

In one embodiment, the present invention provides a method of decreasing the level of ceramide-1-phosphate or inhibits the formation of ceramide-1-phosphate in a cell comprising contacting the cell with a compound that inhibits the activity of ceramide kinase. In another embodiment, the invention relates to a method of reducing the activation of phospholipase A2 in a cell comprising contacting the cell with a compound that inhibits ceramide kinase activity such that the amount of ceramide-1-phosphate in the cell is reduced.

The invention also provides a method of identifying a compound that specifically binds to ceramide kinase comprising: (a) contacting ceramide kinase or a fragment thereof with a compound under conditions conducive to binding between said ceramide kinase or fragment and the compounds; and (b) identifying a compound that specifically binds to said ceramide kinase or fragment thereof.

The invention also provides a method for identifying compounds that modulate the activity of ceramide kinase gene product or homolog of ceramide kinase gene product comprising: (a) contacting a test compound with an organism or a cell containing ceramide kinase gene product or homolog of ceramidase; and (b) comparing the phenotype of the organism or cell with the phenotype of organism or cell that did not contact the test compound, wherein a change in phenotype indicates that the test compound is capable of modulating the activity of ceramide kinase gene product or homolog of ceramidase gene product. Examples of such compounds that inhibit ceramide kinase are (2R,3S,4E) L-erythro-C6-ceramide and (2R,3S,4E) L-erythro-urea-C6-ceramide as shown in FIG. 8.

The invention also provides a method for identifying compounds that modulate ceramide kinase gene expression comprising: (a) contacting a test compound with a cell or cell lysate containing an expressible ceramide kinase gene construct; and (b) detecting the transcription and/or translation of the ceramide kinase gene.

The invention further provides a method of identifying a compound that modulates the activity of ceramide kinase. The method comprising contacting ceramide kinase or a functional fragment thereof with a compound under conditions conducive to binding between the compound and the enzyme, and determining the activity of the ceramide kinase.

The invention also provides a method for identifying compounds that modulate ceramide kinase gene expression, comprising: (a) contacting a test compound with a cell or cell lysate containing a reporter gene operatively associated with a ceramide kinase gene regulatory element; and (b) detecting expression of the reporter gene product.

Various reagents used in the treatment and/or diagnosis of diseases or disorders associated with ceramide kinase activity are provided, as are reagents used in screening assays for compounds that modulate ceramide kinase activity or expression. Accordingly, the present invention provides uses of nucleotide sequences of ceramide kinase genes, including, the human ceramide kinase gene and homologs in other species, and amino acid sequences of ceramide kinases. For example, specific uses of ceramidase kinase nucleic acids described in Sugiura, M., Kono, K., Liu, H., Shimizugawa, T., Minekura, H., Spiegel, S., and Kohama, T. (2002) J. Biol. Chem. 277, 23294-23300 is contemplated. The use of nucleic acids hybridizable to or complementary to the foregoing nucleotide sequences are also provided. The nucleotide sequences can be genomic DNA, cDNA or RNA, including RNAi molecules. The invention also provides uses of a nucleic acid comprising a nucleotide sequence encoding a fragment of a ceramide kinase that displays one or more functional activities of ceramide kinase. The nucleic acid sequence of the human ceramide kinase mRNA is given the GenBank accession number AB079066 (SEQ ID NO:1) and the protein sequence is given the GenBank accession number BAC01154 (SEQ ID NO:2).

The present invention also provides the uses of ceramide kinase antagonists, and particularly the human protein for treatment of conditions related to inflammation. The invention also relates to fragments (derivatives and analogs thereof) of ceramide kinase, which comprise one or more domains of a ceramide kinase protein that can be used for the production of antibodies against ceramide kinase as a ceramide kinase antagonist. Also encompassed are uses of the fragments (derivatives and analogs thereof) of ceramide kinase, which are dominant negative mutants that competitively inhibit a wild-type ceramide kinase protein. These dominant negative mutants have the ability to bind, or compete with ceramide kinase for binding a substrate of ceramide kinase.

The invention also provides a chimeric protein comprising a fragment of a ceramide kinase protein consisting of at least 6, 10, 20, 30, 40, 50, 100, 15, 200, 250, 300, 350, 400, 450, or 480 amino acids fused via a covalent bond to an amino acid sequence of a second polypeptide. In a specific embodiment, the second polypeptide is a signal peptide which facilitates secretion of the chimeric protein so that the protein can be recovered from the culture media.

Antibodies, antisense nucleic acid, RNAi molecules, and ribozyme to ceramide kinase, and ceramide kinase derivatives and analogs, are additionally provided.

Methods of production of the ceramide kinase proteins, derivatives and analogs, e.g., by recombinant means, are also provided.

The present invention also encompasses (a) DNA vectors that contain any of the foregoing ceramide kinase gene, antisense ceramide kinase gene, and modified ceramide kinase gene sequences encoding mutant and fusion ceramide kinase proteins; (b) DNA expression vectors that contain any of the foregoing ceramide kinase gene, antisense ceramide kinase gene, RNAi molecules, and modified ceramide kinase gene sequences encoding mutant and fusion ceramide kinase proteins operatively associated with a regulatory element that directs the transcription and/or expression of the foregoing ceramide kinase gene, antisense ceramide kinase gene, and modified ceramide kinase gene sequences encoding mutant and fusion ceramide kinase proteins; and (c) genetically engineered host cells that contain any of the foregoing DNA vectors or DNA expression vectors.

Accordingly, the compositions of the present invention include cloning vectors, including expression vectors, containing the nucleic acid molecules of the invention, and hosts which contain such nucleic acid molecules. Host cells which comprise such nucleic acid molecules can be used in cell-based drug screening assays.

The present invention provides compositions which include but are not limited to ceramide kinase proteins and analogs and derivatives (including fragments) thereof; antibodies, antisense nucleic acids, RNAi molecules, and ribozymes thereto; nucleic acids encoding the ceramidase proteins, analogs, or derivatives.

Antibodies to the ceramide kinase product can be used as ceramide kinase antagonists to inhibit the activities of ceramide kinase. The methods and compositions of the present invention (i.e., ceramide kinase antagonists) are capable of modulating the level of ceramide kinase gene expression and/or the level of ceramide kinase activity. The methods and compositions of the present invention are also capable of modulating the level of C1P, arachidonic acid, or phospholipase A2. In various embodiments, the ceramide kinase antagonist can also be used in combination with one or more therapeutic agents to treat disease or disorder associated with arachidonic acid release or phospholipase A2 activiation. In specific embodiments, the ceramide kinase antagonist of the present invention may be used to treat inflammatory disorder. In a specific embodiment, the ceramide kinase antagonist of the present invention may be used to treat asthma.

5.1 Screening Assays

In one embodiment, the present invention provides methods for the identification of compounds that, through its interaction with the ceramide kinase gene or ceramide kinase gene product, provide a therapeutic benefit to the recipient, especially one who suffers from cancer, cardiovascular disease, or inflammatory conditions.

The assays of the invention are designed to identify: (i) compounds that modulate the level of ceramide kinase gene expression; (ii) compounds that modulate the level of ceramide kinase activity; (iii) compounds that bind to ceramide kinase gene products including mammalian and non-mammalian homologs of ceramide kinase; and (iv) compounds that interfere with the interaction of the ceramide kinase gene product, including mammalian and non-mammalian homologs of ceramide kinase, with other proteins.

Accordingly, the present invention is related to a method of identifying a compound that binds to a ceramide kinase protein, or a nucleic acid encoding the protein, comprising: (a) contacting the ceramide kinase protein, or a nucleic acid encoding the protein with one or more compounds under conditions conducive to the binding; and (b) identifying the compound that binds to the ceramide kinase protein, or a nucleic acid encoding the protein. For example, assays that identify compounds that bind to ceramide kinase gene regulatory sequences (e.g., promoter sequences) are contemplated. See e.g., Platt, 1994, J. Biol. Chem. 269:28558-28562, which is incorporated herein by reference in its entirety.

In another embodiment, the present invention is related to a method for identifying compounds that modulate ceramide kinase gene expression comprising: (a) contacting a test compound with a cell or cell lysate comprising an expression construct comprising a ceramide kinase gene; and (b) detecting the transcription or translation of the nucleotide sequence of ceramidase kinase.

In yet another embodiment, the present invention is related to a method for identifying compounds that modulate ceramide kinase gene expression, comprising: (a) contacting a test compound with a cell or cell lysate containing a reporter gene operatively associated with the regulatory element of a ceramidase gene; and (b) detecting expression of the reporter gene product. Any reporter gene known in the art can be used, such as but limited to, green fluorescent protein, β-galactosidase, alkaline phosphatase, chloramphenicol acetyltransferase, etc.

In yet another embodiment, the present invention relates to a method for identifying compounds that modulate the activity of ceramide kinase gene product or homolog of ceramide kinase gene product comprising: (a) contacting a test compound with an organism or a cell containing ceramide kinase gene product or homolog of ceramidase; and (b) comparing the phenotype of the organism or cell with the phenotype of organism or cell that did not contact the test compound, wherein a change in phenotype indicates that the test compound is capable of modulating the activity of ceramide kinase gene product or homolog of ceramidase gene product. Phenotypic changes in the test cells can include the release of AA, the activation of PLA₂ and any other known biological indicators associated with the onset of an inflammatory response.

5.2 Pharmaceutical Compositions and Methods of Administration

The compounds and nucleic acid sequences described herein can be administered to a patient at therapeutically effective doses to treat or prevent diseases and disorder associated with inflammation, cardiovascular disorder, cancer, asthma, and rheumatoid arthritis. A therapeutically effective dose refers to that amount of a compound sufficient to result in a healthful benefit in the treated subject. Formulations and methods of administration that can be employed when the therapeutic composition comprises a nucleic acid are described below.

In certain embodiments, the compounds that inhibit ceramide kinase activity are antibodies to ceramide kinase.

In other embodiments, the compounds that inhibit ceramide kinase activity are RNAi molecules.

In other embodiments, the compounds that inhibit ceramide kinase activity are ceramide analogs, such as, (2R,3S,4E)L-erythro-C6-ceramide and (2R,3S,4E)L-erythro-urea-C6-ceramide as shown in FIG. 8.

5.2.1 Antibodies as Ceramide Kinases Antagonists

In one embodiment, the ceramide kinase antagonist is an antibody, preferably a monoclonal antibody. Antibodies of the invention immunospecifically bind ceramide kinase and inhibit ceramide kinase activity.

The antibodies may be generated against ceramide kinase gene products, variants or fragments thereof. As used herein, ceramide kinase gene refers to (a) a gene containing the DNA sequence of SEQ ID NO:1; (b) any DNA sequence that encodes the amino acid sequence of SEQ ID NO:2; (c) any DNA sequence that hybridizes to the complement of the DNA sequences that encode the amino acid sequence of SEQ ID NO:2, under highly stringent conditions, e.g., hybridization to filter-bound DNA in 0.5 M NaHPO₄, 7% sodium dodecyl sulfate (SDS), 1 mM EDTA at 65° C., and washing in 0.1×SSC/0.1% SDS at 68° C. (Ausubel F. M. et al., eds., 1989, Current Protocols in Molecular Biology, Vol. I, Green Publishing Associates, Inc., and John Wiley & sons, Inc., New York, at page 2.10.3); or (d) any DNA sequence that hybridizes to the complement of the DNA sequences that encode the amino acid sequence of SEQ ID NO:2 under moderately stringent conditions, e.g., washing in 0.2×SSC/0.1% SDS at 42° C. (Ausubel et al., 1989, supra) and encodes a gene product functionally equivalent to an ceramide kinase gene product encoded by sequences of SEQ ID NO:2.

In certain embodiments of the invention, the ceramide kinase antagonist is an antibody against fragments and degenerate variants of DNA sequences of (a) through (d), including naturally occurring variants thereof. A ceramide kinase gene sequence preferably exhibits at least about 80% overall similarity at the nucleotide level to the nucleic acid sequence of SEQ ID NO:1, more preferably exhibits at least about 85-90% overall similarity to the nucleic acid sequence of SEQ ID NO:1 and most preferably exhibits at least about 95% overall similarity to the nucleic acid sequence of SEQ ID NO:1. The ceramide kinase gene sequences are preferably of mammalian origin, and most preferably human. Mammals, include but are not limited to, mice, rats, cats, dogs, cattle, pigs, sheep, guinea pigs and rabbits.

In addition to the ceramide kinase gene sequences described above, homologs of such sequences, exhibiting extensive homology to the ceramide kinase gene product present in other species can be identified and readily isolated, without undue experimentation, by molecular biological techniques well known in the art. Accordingly, the ceramide kinase gene encompasses nucleotide sequences encoding ceramide kinase homologs at other genetic loci within the genome that encode proteins which have extensive homology to the ceramide kinase gene product. These genes can also be identified via similar techniques.

In order to clone a mammalian ceramide kinase gene homolog or variants using isolated human ceramide kinase gene sequences as disclosed herein, such human ceramide kinase gene sequences are labeled and used to screen a cDNA library constructed from mRNA obtained from appropriate cells or tissues (e.g., pancreatic epithelial cells) derived from the organism of interest. With respect to the cloning of such a mammalian ceramide kinase homolog, a mammalian cancer cell cDNA library may, for example, be used for screening. The hybridization and wash conditions used should be of a low stringency when the cDNA library is derived from a different type of organism than the one from which the labeled sequence was derived. Low stringency conditions are well known to those of skill in the art, and will vary predictably depending on the specific organisms from which the library and the labeled sequences are derived. For guidance regarding such conditions see, for example, Sambrook et al., 1989, Molecular Cloning, A Laboratory Manual, Cold Springs Harbor Press, N.Y.; and Ausubel et al., 1989, Current Protocols in Molecular Biology, Green Publishing Associates and Wiley Interscience, N.Y.

The ceramide kinase gene also encompasses nucleic acid molecules encoding mutant ceramide kinase, peptide fragments of ceramide kinase, truncated ceramide kinase, and ceramide kinase fusion proteins. The gene products encoded by these nucleic acid molecules include, but are not limited to, peptides corresponding to particular motifs or domains of ceramide kinase including its active site.

Ceramide kinase antagonists also includes nucleic acid molecules, preferably DNA molecules, that hybridize to, and are therefore the complements of, the DNA sequences (a) through (d), in the preceding paragraph. Such hybridization conditions may be highly stringent or moderately stringent, as described above. In instances wherein the nucleic acid molecules are deoxyoligonucleotides (“oligos”), highly stringent conditions may refer, e.g., to washing in 6×SSC/0.05% sodium pyrophosphate at 37° C. (for 14-base oligos), 48° C. (for 17-base oligos), 55° C. (for 20-base oligos), and 60° C. (for 23-base oligos). These nucleic acid molecules may encode or act as ceramide kinase gene antisense molecules useful, for example, in ceramide kinase gene regulation. With respect to ceramide kinase gene regulation, such techniques can be used to modulate, for example, the level of ceramide kinase product, C1P, arachidonic acid or phospholipase A2. Further, such sequences may be used as part of ribozyme and/or triple helix sequences, also useful for ceramide kinase gene regulation.

In a more specific embodiment, an antibody of the invention immunospecifically binds to the active site of the ceramide kinase and decreases phosphorylation of the substrate. In another specific embodiment, the antibody binds to the substrate binding site of ceramide kinase and inhibits or reduces the extent of the interaction with its substrate.

Antibodies of the invention include, but are not limited to, monoclonal antibodies, synthetic antibodies, recombinantly produced antibodies, multispecific antibodies (including bi-specific antibodies), human antibodies, humanized antibodies, chimeric antibodies, synthetic antibodies, intrabodies, single-chain Fvs (scFv) (e.g., including monospecific and bi-specific, etc.), Fab fragments, F(ab′) fragments, disulfide-linked Fvs (sdFv), intrabodies, and epitope-binding fragments of any of the above. In particular, antibodies used in the present invention include immunoglobulin molecules and immunologically active portions of immunoglobulin molecules, i.e., molecules that contain at least one antigen binding site that immunospecifically binds to ceramide kinase and are antagonists of ceramide kinase (e.g., decrease ceramide kinase-substrate binding, decrease ceramide kinase kinase activity. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Patent Publication Nos. WO 94/04678 and WO 94/25591; U.S. Pat. No. 6,005,079; which are incorporated herein by reference in their entireties). In one embodiment, the present invention provides single domain antibodies comprising two V_{H} domains having the amino acid sequence of any of the V_{H} domains of the ceramide kinase antagonistic antibodies (or an antibody that binds to ceramide kinase and/or decreases ceramide kinase phosphorylation activity, and/or inhibits ceramide kinase-substrate interaction. In another embodiment, the present invention also provides single domain antibodies comprising two V_{H} domains comprising one or more of the V_{H} CDRs of any of the ceramide kinase antagonistic antibodies or an antibody that immunospecifically binds to ceramide kinase and/or decreases ceramide kinase phosphorylation activity, and/or inhibits ceramide kinase-substrate interaction, and/or inhibits ceramide kinase enzymatic activity.

Antibodies of the invention include ceramide kinase intrabodies. Generation of intrabodies is well-known to the skilled artisan and is described, for example, in U.S. Pat. Nos. 6,004,940; 6,072,036; 5,965,371, which are incorporated by reference in their entireties herein. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250, which references are incorporated herein by reference in their entireties. Recombinant molecular biological techniques such as those described for recombinant production of antibodies may also be used in the generation of intrabodies.

The antibodies used in the invention may be from any animal origin including birds and mammals (e.g., human, murine, donkey, sheep, rabbit, goat, guinea pig, camel, horse, or chicken). In a most preferred embodiment, the antibody is human or has been humanized. As used herein, “human” antibodies include antibodies having the amino acid sequence of a human immunoglobulin and include antibodies isolated from human immunoglobulin libraries or from mice that express antibodies from human genes.

The antibodies used in the present invention may be monospecific, bispecific, trispecific or of greater multispecificity. Multispecific antibodies may immunospecifically bind to different epitopes of an ceramide kinase polypeptide or may immunospecifically bind to both an ceramide kinase polypeptide as well a heterologous epitope, such as a heterologous polypeptide or solid support material. See, e.g., International Patent Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The ceramide kinase antagonistic antibodies of the invention or fragments thereof can be produced by any method known in the art for the synthesis of antibodies, in particular, by chemical synthesis or, preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with ceramide kinase (either the fall length protein or a domain thereof, e.g., the extracellular domain) and once an immune response is detected, e.g., antibodies specific for ceramide kinase are detected in the mouse serum, the mouse spleen is harvested and splenocytes isolated. The splenocytes are then fused by well known techniques to any suitable myeloma cells, for example cells from cell line SP20 (available from the ATCC) or NHO cells. Hybridomas are selected and cloned by limited dilution. Hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, monoclonal antibodies can be generated by culturing a hybridoma cell secreting an antibody of the invention wherein, preferably, the hybridoma is generated by fusing splenocytes isolated from a mouse immunized with ceramide kinase or a fragment thereof with myeloma cells and then screening the hybridomas resulting from the fusion for hybridoma clones that secrete an antibody able to bind and antagonize ceramide kinase.

Antibody fragments which recognize specific ceramide kinase epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding V_{H} and V_{L} domains are amplified from animal cDNA libraries (e.g., human or murine cDNA libraries of lymphoid tissues). The DNA encoding the V_{H} and V_{L} domains are recombined together with an scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the V_{H} and V_{L} domains are usually recombinantly fused to either the phage gene III or gene VIII. Phage expressing an antigen binding domain that binds to the ceramide kinase epitope of interest can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177; Kettleborough et al., 1994, Eur. J. Immunol. 24:952-958; Persic et al., 1997, Gene 187:9; Burton et al., 1994, Advances in Immunology 57:191-280; International Application No. PCT/GB91/01134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in International Patent Publication No. WO 92/22324; Mullinax et al., 1992, BioTechniques 12:864; Sawai et al., 1995, AJRI 34:26; and Better et al., 1988, Science 240:1041 (said references incorporated by reference in their entireties).

To generate whole antibodies, PCR primers including V_{H} or V_{L} nucleotide sequences, a restriction site, and a flanking sequence to protect the restriction site can be used to amplify the V_{H} or V_{L} sequences in scFv clones. Utilizing cloning techniques known to those of skill in the art, the PCR amplified V_{H} domains can be cloned into vectors expressing a V_{H} constant region, e.g., the human gamma 4 constant region, and the PCR amplified V_{L} domains can be cloned into vectors expressing a V_{L} constant region, e.g., human kappa or lambda constant regions. Preferably, the vectors for expressing the V_{H} or V_{L} domains comprise an EF-1α promoter, a secretion signal, a cloning site for the variable domain, constant domains, and a selection marker such as neomycin. The V_{H} and V_{L} domains may also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Patent Publication Nos. WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the J_{H} region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Patent Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Medarex (Princeton, N.J.) can be engaged to provide human antibodies directed against a selected antigen using technology similar to that described above.

A chimeric antibody is a molecule in which different portions of the antibody are derived from different immunoglobulin molecules such as antibodies having a variable region derived from a non-human antibody and a human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,816,397, which are incorporated herein by reference in their entirety. Chimeric antibodies comprising one or more CDRs from a non-human species and framework regions from a human immunoglobulin molecule can be produced using a variety of techniques known in the art including, for example, CDR-grafting (EP 239,400; International Patent Publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7:805; and Roguska et al., 1994, PNAS 91:969), and chain shuffling (U.S. Pat. No. 5,565,332). In one embodiment, a chimeric antibody of the invention immunospecifically binds ceramide kinase and comprises one, two, or three V_{L} CDRs having an amino acid sequence of any of the V_{L} CDRs of an antibody of the invention within human framework regions. In another embodiment, a chimeric antibody of the invention immunospecifically binds ceramide kinase and comprises one, two, or three V_{H} CDRs having an amino acid sequence of any of the V_{H} CDRs of an antibody of the invention within human framework regions. In another embodiment, a chimeric antibody of the invention immunospecifically binds ceramide kinase and comprises one, two, or three V_{L} CDRs having an amino acid sequence of any of the V_{L} CDRs of an antibody of the invention and further comprises one, two, or three V_{H} CDRs having an amino acid sequence of any of the V_{H} CDRs of an antibody of the invention within human framework regions. In a preferred embodiment, a chimeric antibody of the invention immunospecifically binds ceramide kinase and comprises three V_{L} CDRs having an amino acid sequence of any of the V_{L} CDRs of an antibody of the invention and three V_{H} CDRs having an amino acid sequence of any of the V_{H} CDRs of an antibody of the invention within human framework regions.

Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., U.S. Pat. No. 5,585,089; and Riechmann et al., 1988, Nature 332:323, which are incorporated herein by reference in their entireties.)

5.2.2 RNA Interference

In another embodiment, the present invention provides ceramide kinase antagonists that are RNA interference (RNAi) molecules. RNAi molecules can be used to decrease ceramide kinase expression.

RNAi is based on the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time. Double-stranded (ds) RNA can be used to interfere with gene expression in mammals that produce a phenotype that is the same as that of a null mutant of ceramide kinase. dsRNA for RNAi can be produced by chemical synthesis, by transcription from short DNA templates or by transcription in vivo from transfected DNA constructs and siRNA expression vectors. Synthetic dsRNA can either be in a standard 21 mer dsRNA format with 2 nucleotide overhangs on either end, or as chemically modified synthetic dsRNA molecules that may offer specificity and stability advantages. Recombinant dicer can also be used to convert large dsRNAs into pools of siRNAs suitable for gene silencing in vitro.

As used herein, an RNAi molecule is a double-stranded or single-stranded polynucleotide that initiates RNA interference in a cell, resulting in a decrease in ceramide kinase gene expression. Preferably, the decrease in expression is specific for the ceramide kinase gene. As used herein, the term short, interfering RNA molecule (siRNA) refers to a double-stranded RNA molecule that forms a RNA-induced silencing complex (RISC) in a cell.

A number of empirical methods have been devised for the design of RNAi molecules used in RNA interference. In one example, the first step in designing an appropriate insert is to choose the siRNA target site. siRNA target sites are typically chosen by scanning an mRNA seequence for the dinucleotide Adenosine-Adenosine, recording the 19 nucleotides immediately downstream of the dinucleotide, and then comparing the potential siRNA target sequences with an appropriate genome datebase to eliminate any sequences with significant homology to other genes. Computer algorithms can be used to generate a highly effective sequence, see Example in section 6. Harborth et al. (2003) Antisense Nucleic Acid Drug Dev. 13: 83-106; Khvorova et al. (2003) Cell 115: 209-216; Elbashir et al. (2001) EMBO J. 20: 6877-6888.

In various embodiment, a RNAi molecule of the invention is a nucleic acid molecule that comprises a portion of the nucleic acid sequence of the ceramide kinase gene, and can be a double-stranded DNA molecule, a double-stranded RNA molecule, a single-stranded DNA molecule, a single-stranded RNA molecule, or a double-stranded DNA-RNA hybrid.

In one embodiment, the RNAi molecule is a double-stranded RNA molecule that comprises, independently in each strand, at least 16, 17, 18, 19, 20, 21, 22, 23, 24, 25, 26, 27, 28, 29, or 30 ribonucleotides. In a specific embodiment, the RNAi molecule is a siRNA.

In another embodiment, the RNAi molecule is a polynucleotide which can serve as a template to produce, by the action of a DNA-dependent RNA polymerase or a RNA-dependent RNA polymerase, a RNA molecule a portion of which, forms a strand or a portion of a siRNA. In a specific embodiment, the RNAi molecule features regulatory elements that enable inducible action of a RNA polymerase. In a specific embodiment, the RNAi molecule comprises a hairpin structure. In another specific embodiment, the bases on each strand of a double-stranded RNAi molecule are perfectly complementary.

RNAi molecules including siRNA can be delivered using a number of different methods. The method of delivery depends on whether the RNAi molecules are for cell-based (in vitro) experiments or (in vivo) animal experiments and treatment of a subject. Delivery of siRNAs can be accomplished by: delivery as naked RNA molecules, delivery via plasmid vectors, delivery as short hairpin RNAs (shRNAs), delivery via lentiviral or other retroviral vectors, delivery via vectors, delivery by injection, delivery by transfection, and delivery in a tissue-specific manner. Examples of human cell vectors are discussed in Paul et al. (2002) Nat. Biotechnol. 20: 505-508; Lee et al. (2002) Nat. Biotechnol. 20: 500-505; Brummelkamp et al. (2002) Cancer Cell. 2(3):243-7; Stewart et al. (2003) RNA 9(4):493-501; Wiznerowicz et al. (2003) J Virol. 77(16):8957-61.

For some cell lines, transfection or electroporation can be utilized to achieve stable knockdowns with RNAi molecules. Transfection does not entail the time required for construction of viral vectors. Many transfection reagents combine two components, a novel cationic lipid formulation for RNAI molecule binding and with a neutral lipid, such as dioleoylphosphatidyl-ethanolamine (DOPE), to allow escape from the endosome when the complex is within the cell. Other transfection reagents combine a cellular protein with a polyamine. For delivery of RNAi molecules in vivo, the following methods can be used: (i) RNAi molecules were injected under hydrostatic pressure into the vein of an animal, and silencing of reporter transgenes was observed in different tissues; (ii) direct injection of synthetic modified RNAi into tumors. To improve the delivery of RNAi molecules, the molecules can be coated with protective lipid molecules, such as liposomes, and by attaching ligands that target specific cells to increase the specificity of delivery. Chemically modified synthetics may offer better stability and longer duration of effectiveness than standard RNAi molecules for in vivo applications. Various aspects of RNAi are discussed further in Dillin (2003) Proc. Natl. Acad. Sci. USA 100: 6289-91; Karpilow et al. (2004) PharmaGenomics 32-40; Koppal (2003) Drug Discovery and Development; Novina et al. (2004) Nature 430:161-164; Scherer et al. (2003) Nat. Biotechnol. 21:1457-1465; Scherer et al.(2004) Biotechniques 36:557-561; Schutze (2004) Mol. Cell. Endocrinol. 213:115-119; Tuschl (2002) Nature Biotechnol. 20:446-447.

5.2.3 Effective Dose

Toxicity and therapeutic efficacy of compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

The effective dose of antagonist to be administered during a treatment cycle ranges from about 0.01 to 0.1, 0.1 to 1, or 1 to 10 mg/kg/day. The dose of antagonist to be administered can be dependent on the mode of administration. For example, intravenous administration of an antagonist would likely result in a significantly higher full body dose than a full body dose resulting from a local implant containing a pharmaceutical composition comprising an antagonist. In one embodiment, an antagonist is administered subcutaneously at a dose of 0.01 to 10 mg/kg/day. In another embodiment, an antagonist is administered intravenously at a dose of 0.01 to 10 mg/kg/day. In yet another embodiment, an antagonist is administered locally at a dose of 0.01 to 10 mg/kg/day. It will be evident to one skilled in the art that local administrations can result in lower total body doses. For example, local administration methods such as intradermal administration, or implantation, can produce locally high concentrations of antagonist, but represent a relatively low dose with respect to total body weight. Thus, in such cases, local administration of antagonist is contemplated to result in a total body dose of about 0.01 to 5 mg/kg/day. In yet another embodiment, a particularly high dose of antagonist, which ranges from about 10 to 50 mg/kg/day, is administered during a treatment cycle.

Moreover, the effective dose of a particular antagonist may depend on additional factors, including the type of disease, the disease state or stage of disease, the molecule's toxicity, the molecule's rate of uptake by cells, as well as the weight, age, and health of the individual to whom the antagonist is to be administered. Because of the many factors present in vivo that may interfere with the action or biological activity of an antagonist, one of ordinary skill in the art can appreciate that an effective amount of an antagonist may vary for each individual.

Additionally, the dose of an antagonist may vary according to the particular antagonist used. The dose employed is likely to reflect a balancing of considerations, among which are stability, localization, cellular uptake, and toxicity of the particular antagonist. For example, a particular chemically modified antagonist may exhibit greater resistance to degradation, or may exhibit higher affinity for the target nucleic acid, or may exhibit increased uptake by the cell or cell nucleus; all of which may permit the use of low doses. In yet another example, a particular chemically modified antagonist may exhibit lower toxicity than other antagonists, and therefore can be used at high doses. Thus, for a given antagonist, an appropriate dose to administer can be relatively high or relatively low. Appropriate doses would be appreciated by the skilled artisan, and the invention contemplates the continued assessment of optimal treatment schedules for particular species of antagonist. The daily dose can be administered in one or more treatments.

The antagonist should be delivered to cells which express the ceramide kinase gene in vivo. A number of methods have been developed for delivering antagonists to cells; e.g., the molecules can be injected directly into the tissue site, or modified antagonists, designed to target the desired cells (e.g., antagonists linked to peptides or antibodies that specifically bind receptors or antigens expressed on the target cell surface) can be administered systemically. Antagonists can be delivered to the desired cell population via a delivery complex. In certain embodiments, the delivery complex comprises a targeting means including a sterol, a lipid, a virus, and a target cell specific binding agent. In a specific embodiment, pharmaceutical compositions comprising antagonist are administered via biopolymers, liposomes, microparticles, or microcapsules. In various embodiments of the invention, it may be useful to use such compositions to achieve sustained release of the antagonists. In a specific embodiment, it may be desirable to utilize liposomes targeted via antibodies to specific identifiable antigens.

However, it is often difficult to achieve intracellular concentrations of an antagonist, such as an antisense RNA molecule or a RNAi molecule, sufficient to suppress translation of endogenous mRNAs. Therefore a preferred approach utilizes a recombinant DNA construct in which the antagonist is placed under the control of a strong pol III or pol II promoter. The use of such a construct to transfect target cells in the patient will result in the transcription of sufficient amounts of antagonist that will form complementary base pairs with the endogenous ceramide kinase gene transcripts and thereby prevent translation of the ceramide kinase gene mRNA.

5.2.4 Formulations and Use

Pharmaceutical compositions for use in accordance with the present invention can be formulated in conventional manner using one or more physiologically acceptable carriers or excipients.

Thus, the compounds and their physiologically acceptable salts and solvents can be formulated for administration by inhalation or insufflation (either through the mouth or the nose) or oral, buccal, parenteral or rectal administration.

For oral administration, the pharmaceutical compositions can take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration the compositions can take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator can be formulated containing a powder mix of the compound and a suitable powder base such as lactose or starch.

The compounds can be formulated for parenteral administration (i.e., intravenous or intramuscular) by injection, via, for example, bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

Any anti-inflammatory therapy (e.g., an anti-inflammatory agent) well-known to one of skill in the art can be used in combination with the compositions and methods of the invention. Non-limiting examples of anti-inflammatory agents include non-steroidal anti-inflammatory drugs (NSAIDs), steroidal anti-inflammatory drugs, beta-agonists, anticholingeric agents, antihistamines (e.g., ethanolamines, ethylenediamines, piperazines, and phenothiazine), and methyl xanthines. Examples of NSAIDs include, but are not limited to, aspirin, ibuprofen, salicylates, acetominophen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

5.2.5 Treatment of Conditions, Diseases and Disorders

The method of the present invention may be used to treat certain conditions, diseases and disorder including but not limited to inflammation, cardiovascular disorder, asthma and rheumatoid arthritis. In certain embodiments of the methods of the present invention, the inflammation is caused by an autoimmune disease. In various embodiments, the inflammation is caused by idiopathic thrombocytopenic purpura (ITP), rheumatoid arthritis (RA), systemic lupus erythrematosus (SLE), autoimmune hemolytic anemia (AHA), scleroderma, autoantibody triggered urticaria, pemphigus, vasculitic syndromes, systemic vasculitis, Goodpasture's syndrome, multiple sclerosis (MS), psoriatic arthritis, ankylosing spondylitis, Sjÿgren's syndrome, Reiter's syndrome, Kowasaki's disease, polymyositis and dermatomyositis.

Other examples of the inflammatory conditions which can be prevented, managed, treated, or ameliorated in accordance with the methods of the invention, include, but are not limited to, asthma, allergic disorders, fibrotic disease (e.g., pulmonary fibrosis), psoraisis, multiple sclerosis, systemic lupus erythrematosis, chronic obstructive pulmonary disease (COPD), encephilitis, inflammatory bowel disease (e.g., Crohn's disease and ulcerative colitis), ischemic reperfusion injury, Gout, Behcet's disease, septic shock, undifferentiated spondyloarthropathy, undifferentiated arthropathy, arthritis, rheumatoid arthritis (juvenile and adult), osteoarthritis, psoriatic arthritis, inflammatory osteolysis, sepsis, meningitis, bites by animals (e.g., insect bites), contact with toxic substances produced by plants and animals, and chronic inflammation resulting from viral or bacteria infections.

### 6. EXAMPLES

6.1 Materials and Methods

Cell Culture: A549 lung adenocarcinoma cells (epithelial-derived) were grown in low glucose DMEM (Invitrogen) supplemented with L-glutamine, 10% (v/v) fetal bovine serum (Invitrogen), 200 U/ml of penicillin G sodium, and 200 μg/ml of streptomycin sulfate. Cells were maintained under 80% confluency and standard incubator conditions (humidified atmosphere, 95% air, 5% CO2, 370 C). For treatment, cells were incubated for 2 hours in low glucose DMEM (Invitrogen) supplemented with L-glutamine, 2% (v/v) fetal bovine serum (Invitrogen), 200 U/ml of penicillin G sodium, and 200 μg/ml of streptomycin sulfate prior to the addition of agonist.

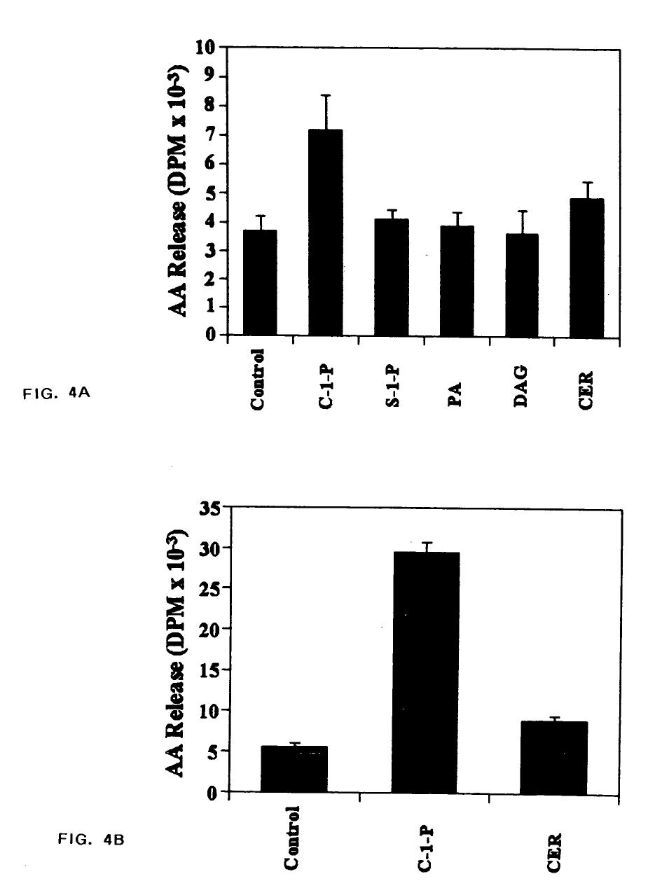

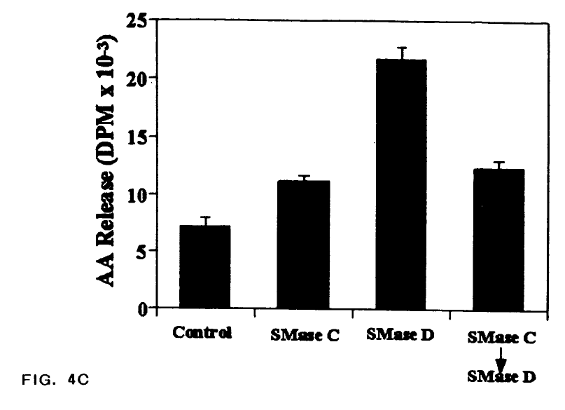

Quantification of arachidonic acid (AA) release: A549 cells (5×10⁴) were labeled overnight with 5 μCi/mL [³H]-AA (5 nM) (NEN). Cells were washed and incubated in DMEM supplemented with 2% fetal bovine serum for 2 hours. Following treatment, media was transferred to 1.5 mL polypropylene tubes, centrifuged at 10,000×g, and ³H-AA cpm determined by scintillation counting. Results were controlled for equivalent number of cells quantified by MTT assay as described (Chalfant, C. E., Rathman, K., Pinkerman, R. L., Wood, R. E., Obeid, L. M., Ogretmen, B., and Hannun, Y. A. (2002) J. Biol. Chem. 277, 12587-12595) and by verification of total AA labeling by scintillation counting. For the experiments in FIG. 4, purified SMase D was obtained from Dr. Alfred H. Merrill, Jr., and SMase C (purified from Bacillus cereus) was obtained commercially from Sigma.

Quantification of prostaglandin E₂ and thromboxane B₂ synthesis: For measurement of PGE₂ and TXB2 levels, aliquots of media were taken at indicated time points and assayed according to manufacturer's instructions using the prostaglandin E₂ and thromboxane B₂ monoclonal EIA Kit from Cayman Chemical. Briefly, media containing PGE₂ or TXB₂ competes with PGE₂ or TXB₂ acetylcholinesterase conjugate for a limited amount of monoclonal antibody. The antibody-PGE₂ or -TXB₂ conjugate binds to a goat-anti-mouse antibody previously attached to the 96-well plate. The plate is washed to remove unbound reagents and then the substrate for acetylcholinesterase is provided. The concentration of each prostanoid in a sample is inversely proportional to the yellow color produced. Results were controlled for equivalent number of cells quantified by MTT assay.

Quantification of ceramide-1-phosphate levels by pulse-labeling with D-e-C₆ NBD-ceramide: Two hours prior to Bligh-Dyer extraction, cells were chased with 20 μM of D-e-C₆ NBD-ceramide (Matreya). At the appropriate times, lipids were extracted by the Bligh-Dyer method, analyzed for NBD-C-1-P formation using TLC analysis, and results normalized to total lipid phosphate as described (Perry, D. K., Bielawska, A., and Hannun, Y. A. (2000) Methods Enzymol. 312, 22-31).

Ceramide Kinase Assay: Ceramide kinase activity was measured as described previously with slight modifications (Bajjalieh, S., and Batchelor, R. (2001) Methods Enzymol. 311, 207-215). Briefly, 2×10⁵ cells were treated with A23187 (1 μg/mL) for 5 minutes or IL-1β (2.5 ng/mL) for 3.5 hours, washed with phosphate buffered saline (ice cold), and resuspended in a 20 mM HEPES (pH 7.2), 50 mM NaCl, 50% glycerol, 1 mM DTT, and protease inhibitors (10 μg/mL each leupeptin, aprotinin, trypsin, chymotrypsin and 1 mM phenylmethylsulfonyl fluoride). Cells were disrupted by two-15 second pulses with a Fisher 550 sonic dismembranator at setting 2. Each sample (10 μg) was assayed for ceramide kinase activity by incubation with NBD-C₁₂ ceramide/cardiolipin/β-octylglucoside and 500 μM ATP in 20 mM HEPES (pH 7.2), 50 mM NaCl, and 1 mM DTT for 15 min at 30° C. Following Bligh-Dyer extraction, lipids were separated by thin layer chromatography, and NBD-C-1-P was visualized/quantified by fluorescent phosphorimaging.

RNAi design and transfections: Knock-downs of ceramide kinase were performed essentially as described using sequence specific siRNA reagents (http:www.mpibpc.gwdg.de/abteilungen/100/105/sirna.html) (Lassus, P., Opitz-Araya, X., and Lazebnik, Y. (2002) Science 297, 1352-1354) using human ceramide kinase RNAi starting 142 nucleotides from start codon (UGCCUGCUCUGUGCCUGUATdTd (SEQ ID NO: 3) and UACAGGCACAGAGCAGGCATdTd (SEQ ID NO:4)). All sequences were evaluated against the database using the NIH blast program to test for specificity (Xeragon, A Qiagen Company). A549 cells (3×10⁴) were transfected with the 21-nucleotide duplexes using OligofectAMINE (Invitrogen) as recommended by the manufacturer. Following the 4 hour transfection, cells were treated and assayed for AA release or PGE₂ synthesis at 26 hours post-transfection.

Reverse transcriptase-polymerase chain reaction (RT-PCR): For evaluating the levels of ceramide kinase mRNA and Ribosomal 18S RNA, 1 μg of total RNA was reverse transcribed using Superscript II reverse transcriptase (Invitrogen) and oligo dT as the priming agent. After one hour incubation at 43.5° C., the reactions were stopped by 70° C. heating for 15 minutes. Template RNA was then removed using RNase H (Invitrogen).

For evaluating ceramide kinase expression, an upstream 5′ primer to ceramide kinase (5′ TCGTCTGTGTCGGCGGAGAT 3′) and a downstream 3′ primer (3′ GCAAGACCCAACCACCGTTTC 5′) were designed to amplify ceramide kinase mRNA crossing at least one exon/intron boundary to eliminate the possibility of amplifying genomic DNA. Using these primers, 10% of the reverse transcriptase reaction was amplified for 15 cycles (94° C., 30 s; 58° C., 30 s; 72° C., 1 min) using Platinum Taq DNA polymerase (Invitrogen). For Ribosomal 18S RNA, primers were obtained from Clontech laboratories, and RNA was amplified for 10 cycles following the standard protocol provided by the company.

6.2 Results

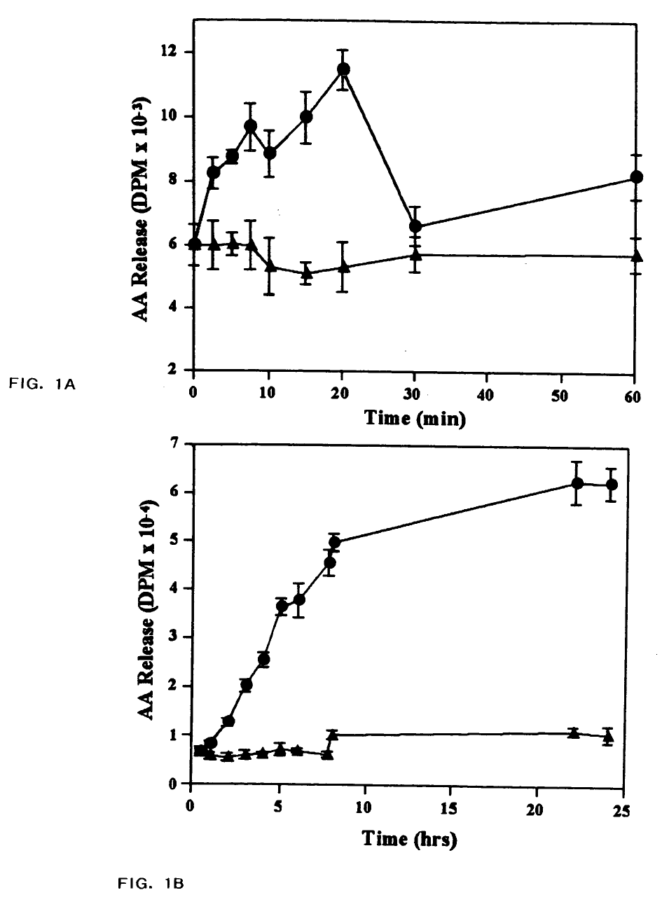

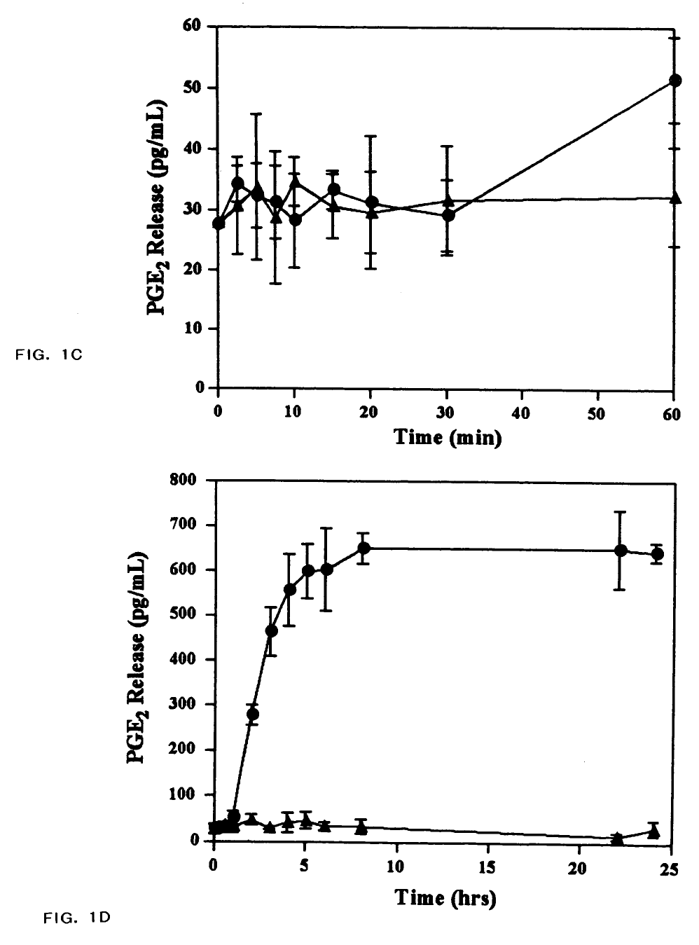

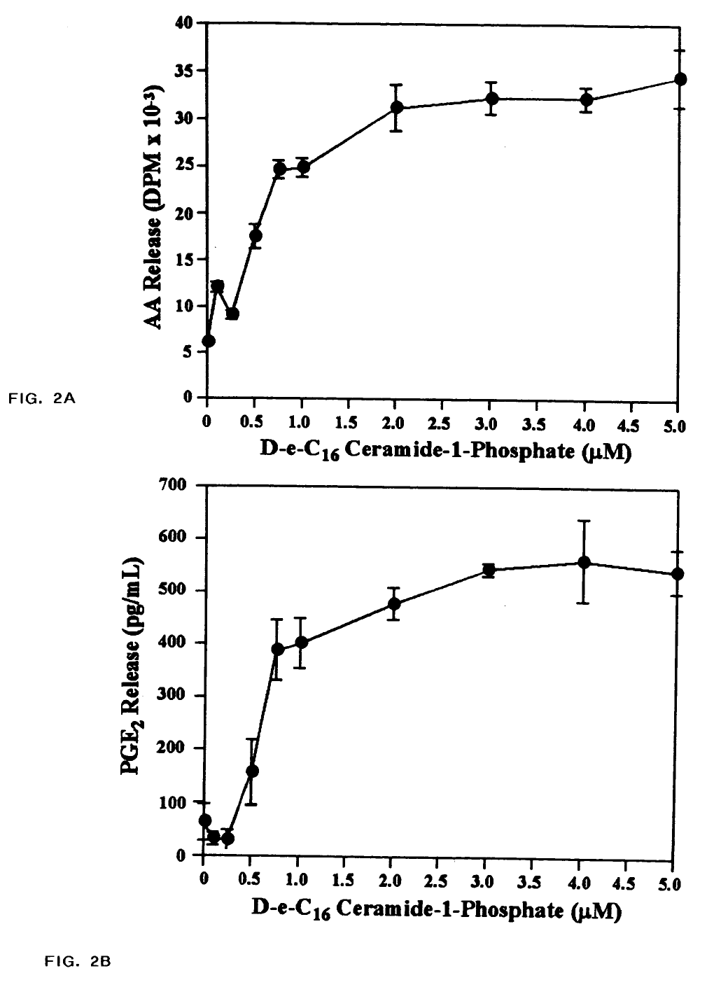

Natural and endogenous ceramide-1-phosphate is a potent and specific inducer of AA release and prostaglandin synthesis in cells. Treatment of A549 cells with D-erythro-C₁₆ ceramide-1-phosphate (2.5 μM), a naturally occurring sphingolipid, induced an increase in AA release within minutes of treatment, with a maximal early increase of approximately 2-fold after 20 minutes, followed by reuptake and a sustained increase in AA release after 1 hour (FIG. 1, Panels A and B). Increased PGE₂ synthesis was observed only after 1 hour, corresponding with the sustained increase in AA liberation (FIGS. 1C and D). This effect of C-1-P on AA release and PGE₂ synthesis was dose-responsive with an EC₅₀ of 400 nM with a concentration as low as 100 nM inducing a significant increase in AA release (FIGS. 2A and B). Therefore, C-1-P induces a time- and dose-dependent increase in both AA and PGE₂ release.

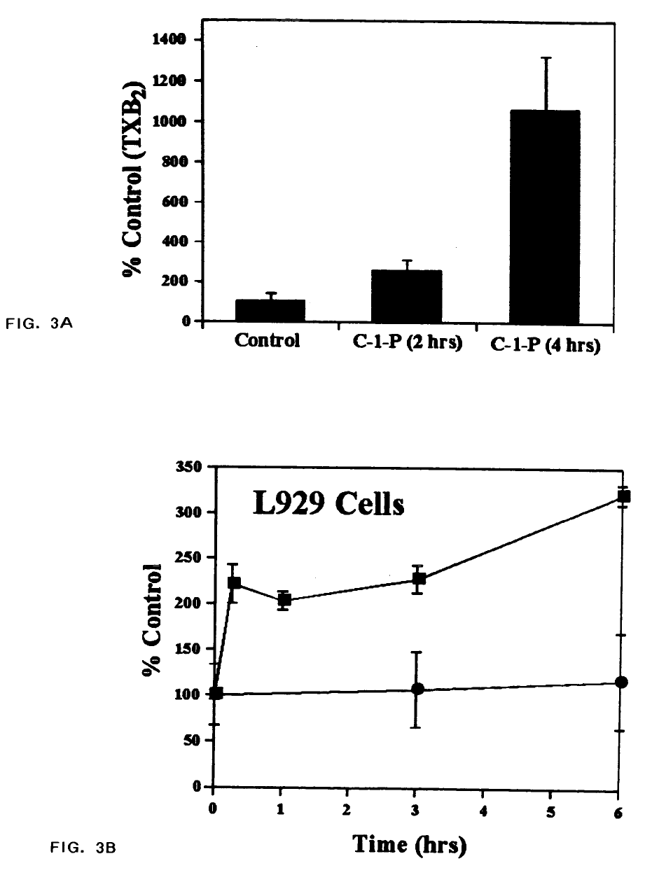

Since C-1-P stimulated AA release, the production of the COX-1 product, thromboxane B₂ (TXB₂), was also examined following C-1-P treatment (Note: A549 cells do not express lipoxygenases or cytochrome P450 (Brinckmann, R., Topp, M. S., Zalan, I., Heydeck, D., Ludwig, P., Kuhn, H., Berdel, W. E., and Habenicht, J. R. (1996) Biochem. J. 318, 305-312; Yamane, M. and Abe, A. J. (2000) Biochemistry 128, 827-835)). C-1-P (1 μM) also dramatically increased TXB₂ by 2.5-fold after 2 hours and 11-fold after 4 hours (FIG. 3A) suggesting that C-1-P induces the activation of a PLA₂ species upstream of cyclooxygenases. This was further demonstrated by using L929 fibroblasts that do not express COX-2. In these cells, PGE₂ synthesis/release was not observed in response to C-1-P (FIG. 3B), but significant AA release of 225% over basal release was detected after 1 hour of C-1-P treatment (1 μM) (FIG. 3B). Therefore, C-1-P induces a time- and dose-dependent increase in both AA and PGE₂ release in A549 cells that express high levels of COX-2, but is specific for AA release, as COX-1 products are produced and cells lacking COX-2 do not produce PGE₂ in response to C-1-P. Furthermore, C-1-P had no effect on the expression of COX-2 as judged by western immunoblotting.

To examine whether the effect of C-1-P on AA synthesis was specific, A549 cells were treated with closely related lipids, di-oleoyl phosphatidic acid (PA), di-oleoylglycerol (DAG), D-erytho-sphingosine-1-phosphate (S-1-P), and D-erythro C₁₆ ceramide (CER) for 2 hours. Only C-1-P induced significant AA release (FIG. 4A). Since CER induced a small increase in AA release, we examined the effects of long term treatment of CER and C-1-P. Unlike C-1-P, CER showed no further increase in AA release (FIG. 4B). Thus, the effect of C-1-P on AA release in cells is lipid-specific.

To determine if endogenously produced C-1-P can induce AA release, A549 cells were treated with SMase D, which hydrolyzes membrane sphingomyelin (SM) to generate C-1-P. SMase D treatment resulted in an approximately 3-fold increase in AA release (FIG. 4C). To demonstrate that the enhancement of AA release was a specific effect of C-1-P generation in response to SMase D, A549 cells were treated with SMase C, which hydrolyzes membrane SM to generate ceramide directly and remove the substrate for SMase D (FIG. 4C) (Feldhaus, M. J., Weyrich, A. S., Zimmerman, G. A., and McIntyre, T. M. (2002) J. Biol. Chem. 277, 4285-4293). SMase C alone only induced modest AA release, which could be the result of the action of ceramide or further conversion to C-1-P (Modur, V., Zimmerman, G. A., Prescott, S. M., and McIntyre, T. M. (1996) J. Biol. Chem. 271, 13094-13102). Importantly, pre-treatment with SMase C inhibited AA release in response to SMase D (FIG. 4C), demonstrating that SM is required for the action of SMase D and that C-1-P is the primary product of SM hydrolysis responsible for the action of SMase D on AA release. Thus, C-1-P is a potent effector of AA release in A549 cells and more importantly, generation of endogenous C-1-P can induce significant AA release.

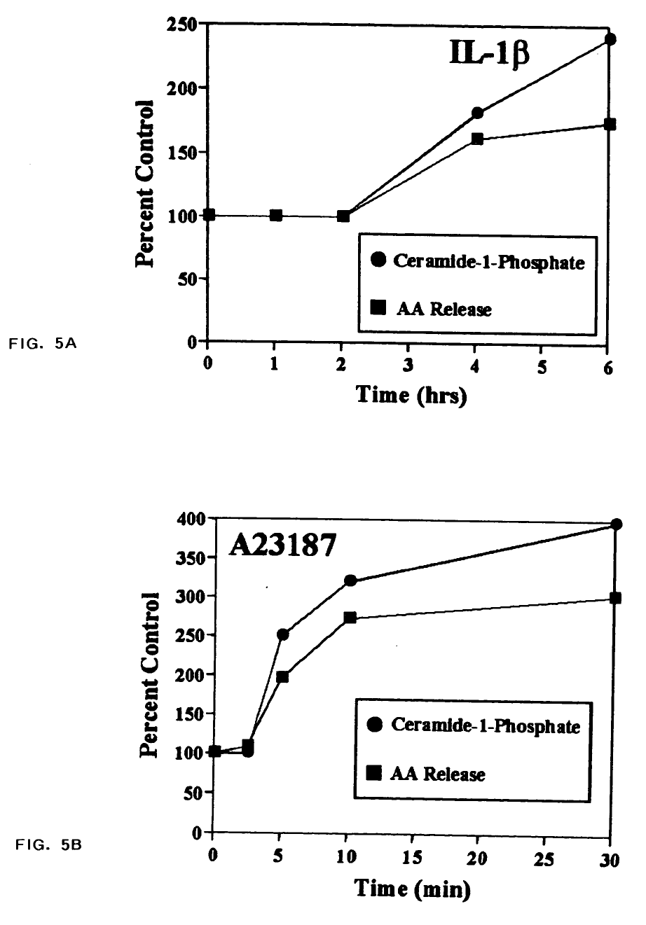

The generation of endogenous ceramide-1-phosphate and activation of ceramide kinase coincides with AA release in response to IL-1, and A23187. If C-1-P functions as a signaling molecule in the pathway to AA release and eicosanoid synthesis, then an increase in the levels of C-1-P should be observed prior to or coinciding with AA release in response to agonists. The cytokine, IL-1β and the calcium ionophore, A23187, are potent and well-established inducers of AA release (Evans, J. H., Spencer, D. M., Zweifach, A., and Leslie, C. C. (2001) J. Biol. Chem. 276, 30150-30160; Perisic, O., Paterson, H. F., Mosedale, G., Lara-Gonzalez, S., and Williams, R. L. (1999) J. Biol. Chem. 274, 14979-14987). IL-1β began to increase AA release after 3.5 to 4 hours of treatment in A549 cells, whereas A23187 increased AA release within minutes (FIGS. 5A and 5B). Using pulse labeling with NBD-D-e-C₆ ceramide, it was found that IL-1β invoked a significant increase of 242% in C-1-P levels after 4 hours (FIG. 5A). A23187 invoked a significant increase in C-1-P levels within 5 minutes of treatment, reaching a maximal increase in C-1-P levels of approximately 4-fold after 10 minutes (FIG. 5B). Thus, IL-1β and A23187 increase C-1-P levels within the relevant time frame of AA release.

Little is known about the biosynthesis of C-1-P in vivo. C-1-P could be generated through either the action of a SMase D type enzyme or ceramide kinase. Mammalian cells are known to contain ceramide kinase activity, which has been recently identified by molecular cloning (Sugiura, M., Kono, K., Liu, H., Shimizugawa, T., Minekura, H., Spiegel, S., and Kohama, T. (2002) J. Biol. Chem. 277, 23294-23300); however, no mammalian SMase D like activity has been described or identified. Moreover, the preceding results on the generation of C-1-P are more consistent with the activation of ceramide kinase, since treatment with agonists enhanced the phosphorylation of NBD-ceramide to NBD-C-1-P. The activation and the role of ceramide kinase in agonist-induced prostanoid synthesis were investigated. Treatment of cells with IL-1β (3.5 hours) or A23187 (5 minutes) increased ceramide kinase activity by 187% and 216%, respectively (FIG. 5C). Thus, known agonists of AA liberation also activate ceramide kinase within the relevant time frame of AA release.

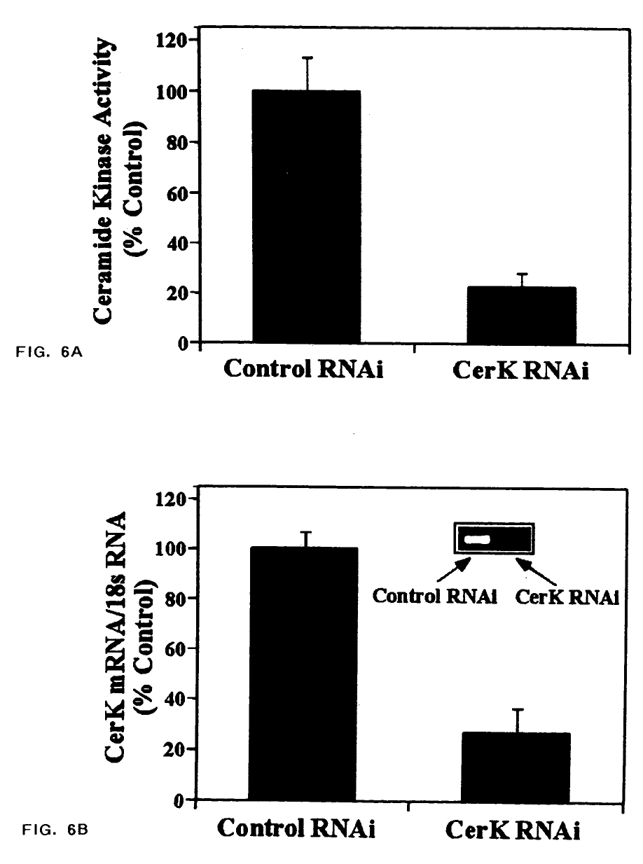

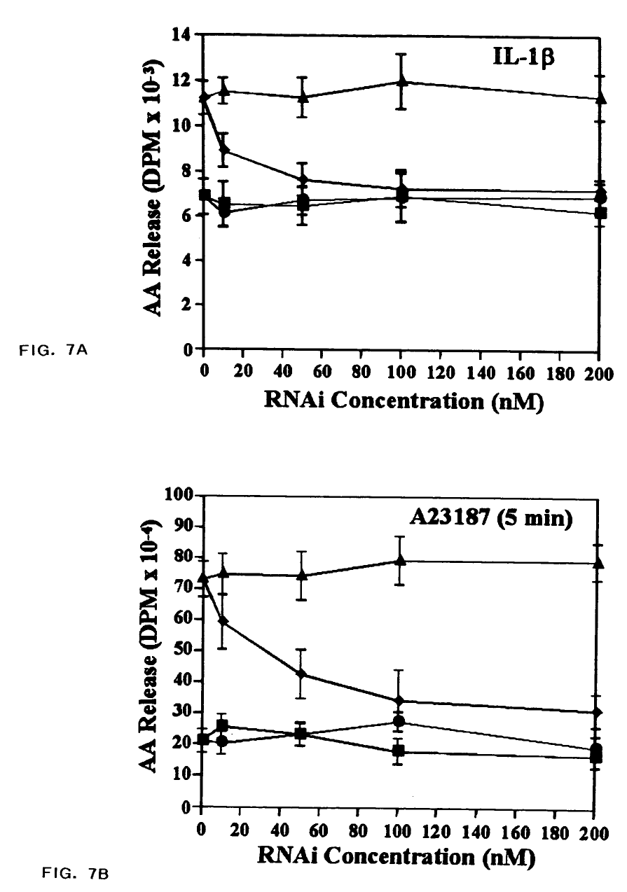

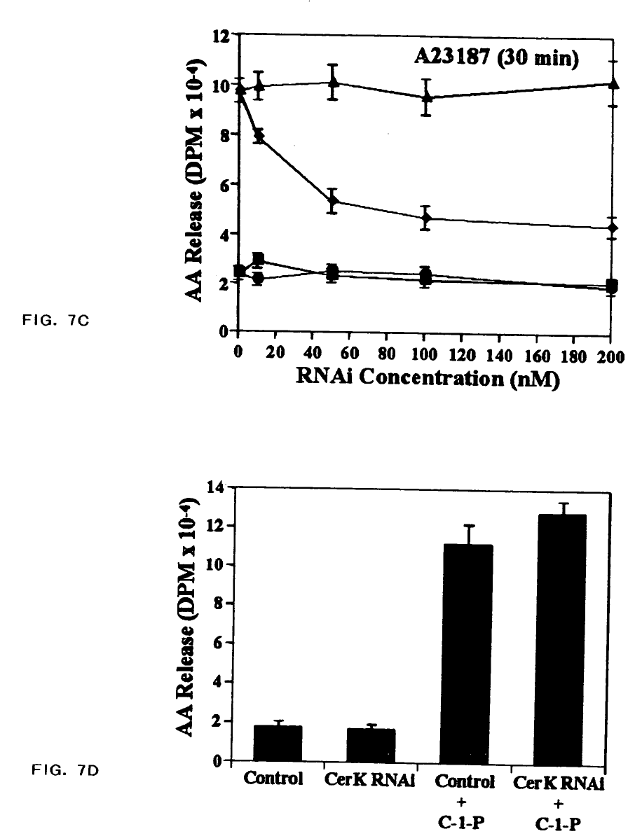

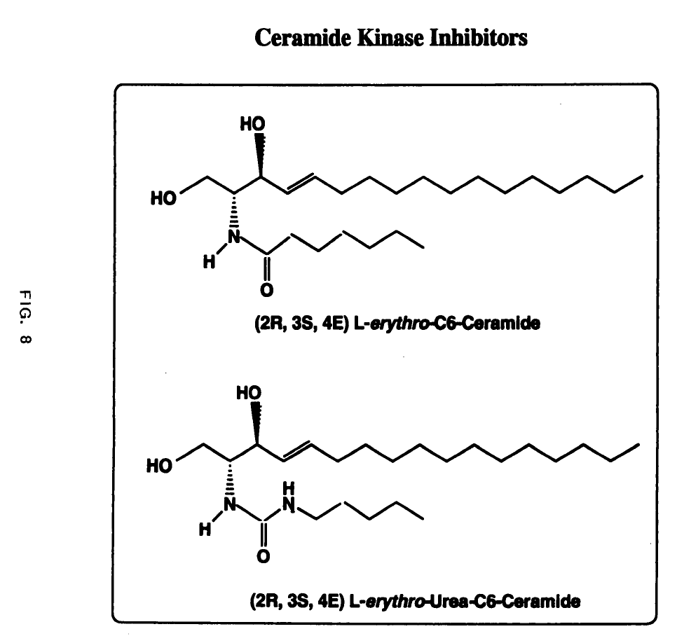

Downregulation of ceramide kinase using RNAi technology inhibits AA release and PGE₂ synthesis in response to IL-1β and A23187. To demonstrate a role for endogenous ceramide kinase in regulating PLA₂ activation/AA release in response to agonists, we used RNAi technology to specifically downregulate ceramide kinase (Lassus, P., Opitz-Araya, X., and Lazebnik, Y. (2002) Science 297, 1352-1354). RNAi specifically decreased the mRNA levels of ceramide kinase by 73%, and endogenous ceramide kinase activity was decreased 67%, without effects on cell number (FIG. 6). Mock transfection and transfection of control (scrambled) RNAi had no effect on the mRNA levels or the activity of ceramide kinase. Although ceramide kinase was cloned because of its similarity to sphingosine kinase 1 (Sugiura, M., Kono, K., Liu, H., Shimizugawa, T., Minekura, H., Spiegel, S., and Kohama, T. (2002) J. Biol. Chem. 277, 23294-23300), there was no decrease in immunoreactive levels of sphingosine kinase-1 observed with RNAi targeted to ceramide kinase. Importantly, downregulation of ceramide kinase by RNAi inhibited AA release in response to A23187 by 72% and AA release in response to IL-1β by 78%. When normalized to the downregulation of ceramide kinase activity and mRNA expression induced by RNAi, this is equivalent to a complete abolishment of AA liberation in response to these agonists. This effect of ceramide kinase RNAi was also dose responsive with an IC₅₀ of approximately 30 nM, indicating the high potency of the action of RNAi (FIGS. 7A, B, and C). Furthermore, ceramide kinase RNAi also inhibited PGE₂ synthesis in response to IL-1β and A23187 to the same extent in A549 cells. In another cell type, L929 fibroblasts, AA release and PGE₂ synthesis in response to IL-1β were also inhibited over 60% by RNAi targeted to ceramide kinase. Ceramide kinase RNAi did not affect the distal “machinery” of PLA₂ activation/AA release as transfection of ceramide kinase RNAi had no effect on AA release in response to C-1-P treatment (FIG. 7D). Thus, ceramide kinase is a necessary enzyme in the induction of AA liberation in response to cytokines and calcium-mobilizing agonists in cells.

6.3 Discussion

The above experiments demonstrate that C-1-P is a potent modulator of AA release and PGE₂ production. Furthermore, endogenous C-1-P and the enzyme that generates C-1-P, ceramide kinase, were necessary for PLA₂ activation in response to cytokines and calcium ionophore. Thus, a specific biology has been established for C-1-P, and for the first time, a physiological role for ceramide kinase, the enzyme that catalyzes the formation of C-1-P, has been determined. Moreover, the results show ceramide kinase to be a potentially important regulatory component of inflammatory responses as AA release is crucial for its conversion to eicosanoids. Therefore, ceramide kinase and C-1-P plays a role in the production of AA.

The sphingomyelin-derived metabolites, ceramide, sphingosine, and sphingosine-1-phosphate are emerging as an important class of bioactive lipid mediators. Although the metabolite of ceramide, C-1-P, was identified more than a decade ago Dressler, K. A., and Kolesnick, R. N. (1990) J. Biol. Chem. 265, 14917-14921; Bajjalieh, S. M., Martin, T. F., and Floor, E. (1989) J. Biol. Chem. 264, 14354-14360; Gomez-Munoz, A., Frago, L. M., Alvarez, L., and Varela-Nieto, I. (1997) Biochem. J. 325,435-440; Gomez-Munoz, A., Duffy, P. A., Martin, A., O'Brien, L., Byun, H. S., Bittman, R., and Brindley, D. N. (1995) Mol. Pharmacol. 47, 833-839; Boudker, O., and Futerman, A. H. (1993) J. Biol. Chem. 268, 22150-22155; Shinghal, R., Scheller, R. H., and Bajjalieh, S. M. (1993) J. Neurochem. 61, 2279-2285; Hinkovska-Galcheva, V. T., Boxer, L. A., Mansfield, P. J., Harsh, D., Blackwood, A. and Shayman, J. A. (1998) J. Biol. Chem. 273, 33203-33209; Hogback, S., Leppimaki, P., Rudnas, B., Bjorklund, S., Slotte, J. P., and Tomquist, K. (2003) Biochem. J. 370, 111-119; Rile, G., Yatomi, Y., Takafuta, T., and Ozaki, Y. (2003) Acta Haematol. 109, 76-83; Carpio, L. C., Stephan, E., Kamer, A., and Dziak, R. (1999) Prostaglandins Leukot. Essential Fatty Acids 61, 267-273). Micromolar concentrations of C-1-P could induce an increase in DNA synthesis in cells (Gomez-Munoz, A., Frago, L. M., Alvarez, L., and Varela-Nieto, I. (1997) Biochem. J. 325, 435-440; Gomez-Munoz, A., Duffy, P. A., Martin, A., O'Brien, L., Byun, H. S., Bittman, R., and Brindley, D. N. (1995) Mol. Pharmacol. 47, 833-839; Hinkovska-Galcheva, V. T., Boxer, L. A., Mansfield, P. J., Harsh, D., Blackwood, A. and Shayman, J. A. (1998) i J. Biol. Chem. 273, 33203-33209; Rile, G., Yatomi, Y., Takafuta, T., and Ozaki, Y. (2003) Acta Haematol. 109, 76-83. A biological role for ceramide kinase in the phagocytosis pathway of neutrophils remains to be defined. The experiments associated with the present invention establishes a biological role for C-1-P as a proximal mediator of AA liberation and is a bioactive sphingolipid metabolites. In contrast to previous reports on C-1-P, C-1-P is found to induce a biological effect (liberation of AA) in the nanomolar range, and the generation of endogenous C-1-P by the action of SMase D alone was sufficient to elicit this biological response in cells. For the cloning of ceramide kinase, see Sugiura, M., Kono, K., Liu, H., Shimizugawa, T., Minekura, H., Spiegel, S., and Kohama, T. (2002) J. Biol. Chem. 277,23294-23300. For the first time, a biological role for ceramide kinase has been established. Therefore, the product of ceramide kinase, C-1-P, fulfills several of the following key criteria of a bioactive lipid. First, C-1-P levels are regulated in response to agonists. Second, exogenous C-1-P induces a specific biochemical and cellular response (release of AA) and this action of C-1-P demonstrates lipid specificity. Third, endogenous C-1-P reproduces these effects specifically. Lastly, the generation of C-1-P is required for AA release in response to inflammatory agonists. Since the experiments presented herein has identified C-1-P as a new messenger lipid in biological systems, the search for a direct intracellular target of C-1-P is encompassed in the invention.

For studies on C-1-P, see Klapisz, E., Masliah, J., Bereziat, G., Wolf, C., and Koumanov, K. S. (2000) J. Lipid Res. 41, 1680-1688; Kitatani, K., Oka, T., Murata, T., Hayarna, M., Akiba, S., and Sato, T. (2000) Arch. Biochem. Biophys. 382, 296-302. The present inventors have found that A23187 induces the activation of ceramide kinase and the accumulation of C-1-P, and since C-1-P is the actual mediator of AA liberation, the conversion of ceramide to C-1-P produces a synergistic effect between ceramide generation and enhanced cPLA₂ activation. For studies on ceramide kinase, see Bajjalieh, S., and Batchelor, R. (2001) Methods Enzymol. 311, 207-215; Reynolds L. J., Hughes L. L., Louis, A. I., Kramer R. M., and Dennis E. A. (1993) Biochim Biophys Acta. 1167, 272-80; Nalefski, E. A., Sultzman, L. A., Martin, D. M., Kriz, R. W., Towler, P. S., Knopf, J. L., and Clark, J. D. (1994) J. Biol. Chem. 269,18239-18249; Clark, J. D., Lin, L. L., Kriz, R. W., Ramesha, C. S., Sultzman, L. A., Lin, A. Y., Milona, N., and Knopf, J. L. (1991) Cell 65,1043-1051; Kramer, R. M., and Sharp, J. D. (1997) FEBS Lett. 410, 49-53; Sharp, J. D., White, D. L., Chiou, X. G., Goodson, T., Gamboa, G. C., McClure, D., Burgett, S., Hoskins, J., Skatrud, P. L., Sportsman, J. R., Becker, G. W., Kang, L. H., Roberts, E. F., and Kramer, R. M. (1991) J. Biol. Chem. 266, 14850-14853; Leslie, C. C. (1997) J. Biol. Chem. 272, 16709-16712; Tay, A., Simon, J. S., Squire, J., Hamel, K., Jacob, H. J., and Skorecki, K. (1995) Genomics 26, 138-141; Hogback, S., Leppimaki, P., Rudnas, B., Bjorklund, S., Slotte, J. P., and Tomquist, K. (2003) Biochem. J. 370, 111-119.

C-1-P indirectly activates cPLA₂, and play a role in Ca²⁺-dependent activation of cPLA₂ and the two other reported mechanisms of cPLA₂ activation, phosphorylation of Ser505 and 727, and PtdIns (Oshima, M., Dinchuk, J. E., Kargman, S. L., Oshima, H., Hancock, B., Kwong, E., Trzaskos, J. M., Evans, J. F., and Taketo, M. M. (1996) Cell 87, 803-809; Takaku, K., Sonoshita, M., Sasaki, N., Uozumi, N., Doi, Y., Shimizu, T., and Taketo, M. M. (2000) J. Biol. Chem. 275, 34013-34016), P₂-dependent activation of cPLA₂ (de Carvalho, M. G., McCormack, A. L., Olson, E., Ghomashchi, F., Gelb, M. H., Yates, J. R., and Leslie, C. C. (1996) J. Biol. Chem. 271, 6987-97, Gordon, R. D., Leighton, I. A., Campbell, D. G., Cohen, P., Creaney, A., Wilton, D. C., Masters, D. J., Ritchie, G. A., Mott, R., Taylor, I. W., Bundell, K. R., Douglas, L., Morten, J., and Needham, M. (1996) Eur. J. Biochem. 238, 690-697; Qiu, Z. H., Gijon, M. A., de Carvatho, M. S., Spencer, D. M., and Leslie, C. C. (1998) J. Biol. Chem. 273, 8203-8211; Mosior, M., Six, D. A., and Dennis, E. A. (1998) J. Biol. Chem. 273, 2184-2191; Balsinde, J., Balboa, M. A., Li, W. H., Llopis, J, and Dennis, E. A. (2000) J. Immunol. 164, 5398-5402).

Phosphorylation of Ser505 and 727 is required for both calcium ionophore- and cytokine-induced AA release (Hefner, Y., Borsch-Haubold, A. G., Murakami, M., Wilde, J. I., Pasquet, S., Schieltz, D., Ghomashchi, F., Yates, J. R., Armstrong, C. G., Paterson, A., Cohen, P., Fukunaga, R., Hunter, T., Kudo, I., Watson, S. P., and Gelb, M. H. (2000) J. Biol. Chem. 275, 37542-37551), and several reports suggest that the MAP kinase pathway is responsible for modulating the phospho-state of cPLA₂ (Gordon, R. D., Leighton, I. A., Campbell, D. G., Cohen, P., Creaney, A., Wilton, D. C., Masters, D. J., Ritchie, G. A., Mott, R., Taylor, I. W., Bundell, K. R., Douglas, L., Morten, J., and Needham, M. (1996) Eur. J. Biochem. 238, 690-697; Zhou, H., Das, S., and Murthy, K. S. (2003) Am. J. Physiol. Gastrointest. Liver Physiol. 284, G472-G480; Lin, L. L., Wartmann, M., Lin, A. Y., Knopf, J. L., Seth, A., and Davis, R. J. (1993) Cell 72, 269-278; Carpio, L. C., Stephan, E., Kamer, A., and Dziak, R. (1999) Prostaglandins Leukot. Essential Fatty Acids 61, 267-273). The inhibitor of serine-threonine protein phosphatases, okadaic acid, has also been shown to induce AA release via cPLA₂ in a calcium-independent manner possibly through effects on the phospho-state of the enzyme (de Carvalho, M. G., McCormack, A. L., Olson, E., Ghomashchi, F., Gelb, M. H., Yates, J. R., and Leslie, C. C. (1996) =i J. Biol. Chem. 271, 6987-97). Okadaic acid mimic the intracellular role of endogenously generated C-1-P since C-1-P is a potent inhibitor (IC₅₀=50 nM) of protein phosphatase-1 and protein phosphatase 2A. Thus, the generation of C-1-P acts indirectly to induce/enhance the phosphorylation of Ser505 and Ser727 of cPLA₂ by inhibiting protein phosphatases and simultaneous activation of the MAP kinase pathway.

Ceramide kinase and C-1-P in cPLA₂ activation also play a role in PtdIns (Oshima, M., Dinchuk, J. E., Kargman, S. L., Oshima, H., Hancock, B., Kwong, E., Trzaskos, J. M., Evans, J. F., and Taketo, M. M. (1996) Cell 87, 803-809; Takaku, K., Sonoshita, M., Sasaki, N., Uozumi, N., Doi, Y., Shimizu, T., and Taketo, M. M. (2000) J. Biol. Chem. 275, 34013-34016; Sugiura, M., Kono, K., Liu, H., Shimizugawa, T., Minekura, H., Spiegel, S., and Kohama, T. (2002) J. Biol. Chem. 277, 23294-23300. C-1-P also play a role in the activation of PGE₂, neurotransmitters, and airway epithelial inflammation since the highest levels of C-1-P are found in synaptic vesicles (Bajjalieh, S., and Batchelor, R. (2001) Methods Enzymol. 311, 207-215; Bajjalieh, S. M., Martin, T. F., and Floor, E. (1989) J. Biol. Chem. 264, 14354-14360).

The present invention is not to be limited in scope by the specific embodiments described herein. The specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims.

Various references are cited herein, the disclosures of which are incorporated by reference in their entireties.

What is claimed:

1. A method of inhibiting the production of ceramide-1-phosphate in a cell comprising delivering to the cell an effective amount of a ceramide kinase antagonist.

2. The method of claim 1 wherein said ceramide kinase antagonist is an antibody that immunospecifically binds to ceramide kinase.

3. The method of claim 1 wherein said ceramide kinase antagonist is a RNAi molecule.

4. The method of claim 3 wherein said RNAi molecule is a siRNA.

5. The method of claim 3 wherein said RNAi molecule comprises the nucleic acid sequence of SEQ ID NO:3, SEQ ID NO:4, or both.

6. The method of claim 1 wherein said ceramide kinase antagonist is a ceramide analog.

7. The method of claim 6 wherein said ceramide analog is (2R,3S,4E) L-erythro-C6-ceramide or (2R,3S,4E)L-erythro-urea-C6-ceramide.

8. The method of claim 1 wherein the ceramide kinase antagonist is administered via a delivery complex comprising a targeting means including a sterol, a lipid, a virus, or a target cell specific binding agent.

9. A method of treating a condition related to activation of phospholipase A2 in a subject comprising delivered to said subject a pharmaceutical composition comprising an effective amount of a ceramide kinase antagonist.

10. The method according to claim 9 in which the condition is inflammation, cardiovascular disorder, cancer, asthma or rheumatoid arthritis.

11. The method of claim 9 wherein said ceramide kinase antagonist is an antibody that immunospecifically binds to ceramide kinase.

12. The method of claim 9 wherein said ceramide kinase antagonist is a RNAi molecule.

13. The method of claim 12 wherein said RNAi molecule is a siRNA.

14. The method of claim 12 wherein said RNAi molecule comprises the nucleic acid sequence of SEQ ID NO:3, SEQ ID NO:4, or both.

15. The method of claim 9 wherein said ceramide kinase antagonist is a ceramide analog.

16. The method of claim 15 wherein said ceramide analog is (2R,3S,4E)L-erythro-C6-ceramide or (2R,3S,4E)L-erythro-urea-C6-ceramide.

17. The method of claim 9 wherein the ceramide kinase antagonist is administered via a delivery complex comprising a targeting means including a sterol, a lipid, a virus, or a target cell specific binding agent.

18. (2R,3S,4E)L-erythro-urea-C6-ceramide.

19. The (2R,3S,4E)L-erythro-urea-C6-ceramide of claim 18 which is purified.

20. A pharmaceutical composition comprising the compound of claim 19.


SEQUENCE LISTING (4 sequences)

SEQ ID NO: 1 - DNA, 4445 bp, Homo sapiens
  Human ceramide kinase
gcgccgcctcccgcagcccgccgcccgccgcccgccgcccgggccactgcaggggccgctaacggtccggcgcccctcggcgtccgcgcgcccccagcctggcggacgagcccggcggcggagatgggggcgacgggggcggcggagccgctgcaatccgtgctgtgggtgaagcagcagcgctgcgccgtgagcctggagcccgcgcgggctctgctgcgctggtggcggagcccggggcccggagccggcgcccccggcgcggatgcctgctctgtgcctgtatctgagatcatcgccgttgaggaaacagacgttcacgggaaacatcaaggcagtggaaaatggcagaaaatggaaaagccttacgcttttacagttcactgtgtaaagagagcacgacggcaccgctggaagtgggcgcaggtgactttctggtgtccagaggagcagctgtgtcacttgtggctgcagaccctgcgggagatgctggagaagctgacgtccagaccaaagcatttactggtatttatcaacccgtttggaggaaaaggacaaggcaagcggatatatgaaagaaaagtggcaccactgttcaccttagcctccatcaccactgacatcatcgttactgaacatgctaatcaggccaaggagactctgtatgagattaacatagacaaatacgacggcatcgtctgtgtcggcggagatggtatgttcagcgaggtgctgcacggtctgattgggaggacgcagaggagcgccggggtcgaccagaaccacccccgggctgtgctggtccccagtagcctccggattggaatcattcccgcagggtcaacggactgcgtgtgttactccaccgtgggcaccagcgacgcagaaacctcggcgctgcatatcgttgttggggactcgctggccatggatgtgtcctcagtccaccacaacagcacactccttcgctactccgtgtccctgctgggctacggcttctacggggacatcatcaaggacagtgagaagaaacggtggttgggtcttgccagatacgacttttcaggtttaaagaccttcctctcccaccactgctatgaagggacagtgtccttcctccctgcacaacacacggtgggatctccaagggataggaagccctgccgggcaggatgctttgtttgcaggcaaagcaagcagcagctggaggaggagcagaagaaagcactgtatggtttggaagctgcggaggacgtggaggagtggcaagtcgtctgtgggaagtttctggccatcaatgccacaaacatgtcctgtgcttgtcgccggagccccaggggcctctccccggctgcccacttgggagacgggtcttctgacctcatcctcatccggaaatgctccaggttcaattttctgagatttctcatcaggcacaccaaccagcaggaccagtttgacttcacttttgttgaagtttatcgcgtcaagaaattccagtttacgtcgaagcacatggaggatgaggacagcgacctcaaggagggggggaagaagcgctttgggcacatttgcagcagccacccctcctgctgctgcaccgtctccaacagctcctggaactgcgatggggaggtcctgcacagccctgccatcgaggtcagagtccactgccagctggttcgactctttgcacgaggaattgaagagaatccgaagccagactcacacagctgagaagccggcgtcctgctcacaaactgggaaagtgtgaaaactatttaagataattattacagaccaattatgttgatatatacatttaaatgtagaaatttatttttgatagttaaatcttgattttagaagaaaacccttttgtcaacaattttgtgtacatatttggcattttcagttctgtacgcatctgcgggttgcagcccacgccgcttactctcagcggatgcagctgctcacttgggggcactggcctcttaggttttaacgatgtcaacagtgtagtttagaaaatggcccgttagtggctctattgcaataatgttagggacattatatgatttccacgcaggtcacaccatctgggcctgaggtagcagtgggtcactttgatccactttgcaggacttattctgtaacggtttgtggccaagttttgggaagtggttgattatctttgccttcatttcaccttcctcttcgtttacggttaggacatcgctgcttgatccttacaatactgtgcaactgcaatgcaacgtggccctgcttcaggtgatccgcgggaggggcctccacgccagcgccgggaaggctgctggggcctccacacctgcctcatcacggcggcgaggctacgacaatccggctgggagcatgaccttggcgtctgttctgggagcacagatgataagctctggaagctggcagtgtgtaaagcactggcaagtttgttactgttaaaatgtcaaataccaatgctttatatcgacgcgaagtgcttaacacagccgggcttgggggcagtcaggaggaagctggccatccgtggaggaggggccggtcctggactcccgcaggactcctctgatgcagggcctgaagtctgtacacgtggtccagatttgtccttgtcttttcttcacactgagttctctatatttattgaacatcttgtccttttaagccagagtagtgtaaactgcgtctcggatgtctgtcttttgtctcgaagccacgatggatcgctggtttcctctgcagcgcaagggctccggcgaccagaggattcttcccggaaggcattcctgccgcgctccccggggcacccctcaattgtgtactacgtccttgtttagtgtgtatccgtgcccacgtagatgatgtctgtaacgcagttttgtttgaaatatgagaatatgcggcttaaactttgatctgtaaggagcggggccgtggccgtttggagcacgctgtagacaccgttcctcatgctgccgggtgggttttgcagaagctcccttagtgatttcacgtttaacaggcagcatccattttcagaatttcctggcattgatttatattttgaagcatacaggaaacttctcgtttcctcgtttagccccacccagatcaggtgaaagggcagctttaatggtggtttttatggaccacattatcagagagcactgtgcaagccaaatggttcaataatgaatgaaaattctgggtgtaaagagtaaatatgccctggctctttctaccaatgtttgctcctggttggaaagaaaccaaagatttaagacgggctgctcttccagactggctgtgcctgcctgtgcccagcaacctgtgcagccggcagtgtgcctggtgtcacgccaggaggctgtggctgctgtgggccctctggaattgtgctcctcacaaagtttccccaaaaggttcttctaagcctttattgtccctggtaaatgtttcccggctgggcgcggtggctcacgcctgtaatcccagcactttgggaggccgaggcgggtggatcacctaaggtcaggagtttgagatcagcctgcccaacatggtgaaacctcgtctctactaaaaatacacaacttagccagtcttgttggcgcacgcctgtaatctcagctactagggacgctgaggcaggagaatcgcttgaacccaagaggtggaggttgcggtgagccaagattgcgccactgcactccagcctgggcaacagagggagactccatcgccccccctcaacaacaaaaaaagtttcccatacactggcctgccccaaaacccactaacaattttagcaaaacagtccaggccaaagaggaagcatttcatgttcaataagaaacccagccattccgcatggctggttcctgagtggctctggtgatactctccagccacctgctgacattcagaatctcagacctcgggactgctgttgcggtaccgtgtgtctgacacctgccagcagccctttgctatctgcgcgcaggatgggggtgactgcccagacattcccgctagataggctctgatttccggggcagcctttcagatgcggcagacatacaacacctgtactttagagttttaagggaaaaaaaaatcagaagtgctggttagatagtaaaaacttaggataacttagaaaggctagttttagcttcctttgtggctccctggtgcaaaacaattagcagttatgcaatggacctgattctagtttattctaattaagaagtgaggccgggtttgacttcgttcctgaatacaatcttgagtaactgggaaagtctgagtgaaaggatggcctcattctctttctaatcttgctggtttcaagattagaaaatggcattatttgatctgaaatgtttgagaagacacgaataaag

SEQ ID NO: 2 - protein, 537 aa, Homo sapiens
  Human ceramide kinase
MGATGAAEPLQSVLWVKQQRCAVSLEPARALLRWWRSPGPGAGAPGADACSVPVSEIIAVEETDVHGKHQGSGKWQKMEKPYAFTVHCVKRARRHRWKWAQVTFWCPEEQLCHLWLQTLREMLEKLTSRPKHLLVFINPFGGKGQGKRIYERKVAPLFTLASITTDIIVTEHANQAKETLYEINIDKYDGIVCVGGDGMFSEVLHGLIGRTQRSAGVDQNHPRAVLVPSSLRIGIIPAGSTDCVCYSTVGTSDAETSALHIVVGDSLAMDVSSVHHNSTLLRYSVSLLGYGFYGDIIKDSEKKRWLGLARYDFSGLKTFLSHHCYEGTVSFLPAQHTVGSPRDRKPCRAGCFVCRQSKQQLEEEQKKALYGLEAAEDVEEWQVVCGKFLAINATNMSCACRRSPRGLSPAAHLGDGSSDLILIRKCSRFNFLRFLIRHTNQQDQFDFTFVEVYRVKKFQFTSKHMEDEDSDLKEGGKKRFGHICSSHPSCCCTVSNSSWNCDGEVLHSPAIEVRVHCQLVRLFARGIEENPKPDSHS

SEQ ID NO: 3 - DNA, 21 bp, Artificial Sequence
  misc_feature 1-19: Chemically synthesized (siRNA sense strand)
ugccugcucugugccuguatt

SEQ ID NO: 4 - DNA, 21 bp, Artificial Sequence
  misc_feature 1-19: Chemically synthesized (siRNA sense strand)
uacaggcacagagcaggcatt